(19) United States
(12) Patent Application Publication
(10) Pub. No.: US 2005/0281829 A1
(43) Pub. Date: Dec. 22, 2005

(54) Fc chimeric proteins with anti-HIV drugs

(76) Inventors: Cristina A. Tan Hehir, Niskayuna, NY (US); Adam R. Mezo, Waltham, MA (US); Robert T. Peters, W. Roxbury, MA (US); James M. Stattel, Leominster, MA (US); Vito J. Palombella, Needham, MA (US); Alan R. Bitonti, Acton, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/841,956

(22) Filed: May 6, 2004

Related U.S. Application Data
(60) Provisional application No. 60/468,835, filed on May 6, 2003.

(51) Int. Cl. A61K 39/42; C07K 16/10

(52) U.S. Cl. 424/160.1; 530/388.3

(57) ABSTRACT

The invention relates to anti viral agents comprised of viral fusion inhibitors and at least a portion of an immunoglobulin constant region. The invention further relates to anti viral agents comprised HIV viral fusion inhibitors and an Fc fragment of an immunoglobulin. The invention also relates to methods of treating a viral infection, including HIV infection.

### DESCRIPTION OF THE INVENTION

This application claims priority to U.S. Provisional Appln. No. 60/468,835, filed on May 6, 2003, which is incorporated by reference.

### FIELD OF THE INVENTION

The invention relates generally to the field of anti viral therapy. More specifically, the invention relates to therapeutic agents specific for Human Immunodeficiency Virus (HIV).

### BACKGROUND OF THE INVENTION

HIV, the etiological agent of Acquired Immune Deficiency Syndrome (AIDS), is an enveloped retrovirus which infects and kills CD4⁺ cells of the immune system. The result of infection is a degenerative disease leaving the infected subject immuno-compromised and susceptible to a variety of opportunistic infections. (Barre-Sinoussi et al. 1983, Science 220:868; Gallo et al. 1984, Science 224:500).

The HIV virion is comprised of an RNA genome encased in a viral protein shell called Gag, which in turn is surrounded by a lipid membrane derived from an infected cell. Inserted into the lipid membrane are two viral envelope proteins, gp120 and gp41. The two envelope proteins originate from cleavage of a common precursor, gp160, and remain non-covalently associated with one another on the virion surface (Hammarskjold et al. 1989, Biochem. Biophys. Acta 989:269). Multiple copies of gp120 and gp41 associate to form oligomers believed to be trimers, which dot the surface of the virion. gp120 mediates viral attachment to susceptible cells by binding receptor proteins expressed on the cell surface, while gp41 mediates post-binding viral fusion of the cellular and viral membranes, thus permitting entry of the viral genome into the cytoplasm of the target cell.

HIV commences infection of a target cell by binding to cell surface receptors. It is believed the HIV envelope protein, gp120, first binds to CD4 (Dagleish et al. 1984, Nature 312:763; Maddon et al. 1986, Cell 47:333). This event induces conformational changes in gp120 permitting gp120 to bind one of two co-receptors, CXCR4 or CCR5. This second binding event triggers additional conformational changes in the gp120/gp41 oligomer resulting in the insertion of gp41 in the target cell membrane, thus initiating fusion of the viral and cellular membranes (Louis et al. 2001, J. Biol. Chem. 31(3):29485).

While gp41 in its active state mediates viral fusion with the target cell, it remains inactive in its native state in the intact cell free virion. The binding events described above trigger conformational changes in the gp41 protein that permit it to transition from an inactive to an active state. It is believed the active conformation of gp41 consists of a trimer of hairpins. The trimer of hairpins consists of six α-helices. Three of these helices are packed in an anti-parallel manner around a central core comprising a three stranded coiled-coil. It is believed that this structure permits the close apposition of the viral and cellular membranes that results in viral fusion and entry into the target cell (Root et al. 2001, Science 291:884; Chan et al. 1998, Cell 93:681).

Therapeutic peptides derived from the amino and carboxy terminus of gp41 have been shown, both in vivo and in vitro, to inhibit fusion of the viral and cellular membranes thereby blocking viral entry or infection of the target cell. These fusion inhibitors are thus potent anti viral therapeutics with demonstrated anti viral activity in the nanomolar and micromolar range. Examples of these therapeutics include T21 (DP-107) (Wild et al. 1992, Proc. Natl. Acad. Sci USA: 89:10541), T20 (DP-178) (Wild et al. 1994, Proc. Natl. Acad. Sci USA: 91: 9770; Kilby et al. 1998, Nat. Med. 4(11):1302; U.S. Pat. No. 5,464,933), and T1249 (De Clerq et al. 2002, Med. Res. Rev. 22(6):531).

It has been suggested that fusion inhibitors act by binding to a pre-hairpin intermediate, thus blocking formation of the fusogenic coiled-coil hairpin structure required for viral fusion (Root et al. 2001, Science 291:884). Fusion inhibitors have been combined with other anti-retroviral drugs such as reverse transcriptase inhibitors and protease inhibitors as part of a therapeutic regimen for controlling HIV infection (U.S. Pat. No. 6,475,491).

While providing potent anti viral activity that targets a novel aspect of the HIV life cycle, fusion inhibitors to date have not been without their shortcomings. Chief among these are the fact that they are chemically synthesized, a process that results in low yields, and is time consuming, difficult and expensive (U.S. Pat. Nos. 5,464,933; 6,015,881; 6,281,331). Additionally, due to its rapid clearance and tendency to bind to human serum albumin, the drug must be administered at high doses and at relatively frequent intervals. Moreover, because the drug consists of a relatively small peptide fragment, and thus is susceptible to digestive enzymes, the fusion inhibitor must be administered parenterally.

Accordingly one aspect of the invention provides more stable HIV chimeric proteins with viral fusion inhibitory activity, with higher bioavailability, and longer half life, that require less frequent administration. An additional aspect of certain embodiments of the invention provides an HIV fusion inhibitor that does not require parenteral administration. Yet another aspect of certain embodiments of this invention provides a faster, more efficient, less expensive, method of making HIV fusion inhibitors.

An aspect of the invention provides for chimeric proteins comprising HIV fusion inhibitors and at least a portion of an immunoglobulin constant region.

### SUMMARY OF THE INVENTION

The invention relates to chimeric proteins with viral fusion inhibitory activity having improved stability, half life and bioavailabilty compared to known viral peptide fusion inhibitors wherein said improved viral fusion inhibitors are comprised of at least one viral fusion inhibitor and at least a portion of an immunoglobulin constant region. The invention thus relates to a chimeric protein comprising at least one viral fusion inhibitor and at least a portion of an immunoglobulin constant region.

In another aspect, the invention relates to a method of treating a subject infected with a virus, said method comprising administering a therapeutically effective amount of a chimeric protein, wherein said chimeric protein comprises at least one viral fusion inhibitor and at least a portion of an immunoglobulin constant region.

In another aspect, the invention relates to a method of inhibiting viral fusion with a mammalian cell comprising combining the mammalian cell with at least one chimeric protein, wherein said chimeric protein comprises at least a portion of an immunoglobulin constant region and at least one viral fusion inhibitor.

In another aspect, the invention relates to a method of making a chimeric protein comprising at least one viral fusion inhibitor and at least a portion of an immunoglobulin constant region, said method comprising transfecting a cell with a DNA construct comprising a first DNA sequence encoding at least a portion of an immunoglobulin constant region operatively linked to a second DNA sequence encoding a viral fusion inhibitor; culturing said cell under conditions such that the chimeric protein is expressed; and isolating said chimeric protein.

In another aspect, the invention relates to a method of making a chimeric protein comprising at least one viral fusion inhibitor and at least a portion of an immunoglobulin constant region, said method comprising transfecting a cell with a DNA construct comprising a DNA sequence encoding at least a portion of an immunoglobulin constant region; culturing said cell under conditions such that the portion of an immunoglobulin constant region is expressed; isolating said portion of an immunoglobulin constant region from said cell; chemically synthesizing a viral fusion inhibitor; reacting the isolated portion of an immunoglobulin constant region with the viral fusion inhibitor to make said chimeric protein.

In another aspect, the invention relates to a nucleic acid molecule said molecule comprising a nucleic acid sequence encoding at least one viral fusion inhibitor and at least a portion of an immunoglobulin constant region.

In another aspect, the invention relates to a nucleic acid construct comprising a DNA sequence encoding at least one viral fusion inhibitor and at least a portion of an immunoglobulin constant region.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1A shows the amino acid sequence of an Fc-T20 construct (SEQ ID NO:4) with a peptide linker between T20 and Fc; T20 is shown in bold and the linker is italicized.

FIG. 1B shows the amino acid sequence of a T20-Fc construct (SEQ ID NO:5) with a peptide linker between T20 and Fc; T20 is shown in bold and the linker is italicized.

FIG. 1C shows the amino acid sequence of an Fc-T20-GS16 (SEQ ID NO:6) construct with a peptide linker between T20 and Fc; T20 is shown in bold and the linker is italicized.

FIG. 1D shows the amino acid sequence of a T20-Fc-GS18 (SEQ ID NO:7) construct with a peptide linker between T20 and Fc, T20 is shown in bold and the linker is italicized.

FIG. 1E shows the amino acid sequence of an Fc-T20-Phe-Cys (SEQ ID NO:8) construct with a peptide linker between T20 and Fc; T20 is shown in bold and the linker is italicized.

FIG. 1F shows the amino acid sequence of an Fc-T20 (SEQ ID NO:9) construct with a peptide linker between T20 and Fc and a His tag and an enterokinase sitelinked to the N terminus of Fc; T20 is shown in bold and the linker is italicized, the His tag is underlined and the enterokinase site is underlined and italicized.

FIG. 2A shows the amino acid sequence of T20 (SEQ ID NO:1).

FIG. 2B shows the amino acid sequence of T21 (SEQ ID NO:2).

FIG. 2C shows the amino acid sequence of T1249 (SEQ ID NO:3).

FIG. 2D shows the amino acid sequence for NccGgP41 (SEQ ID NO:18).

FIG. 2E shows the amino acid sequence of 5 helix (SEQ ID NO:19).

FIG. 3A shows the sequence of an Fc fragment of an IgG (SEQ ID NO:16).

FIG. 3B shows the sequence of an Fc fragment of an IgG (SEQ ID NO:17).

FIG. 4A shows the nucleic acid sequence of an Fc-T20 construct (SEQ ID NO:10) with a peptide linker between T20 and Fc; T20 is shown in bold and the linker is italicized.

FIG. 4B shows the nucleic acid sequence of a T20-Fc construct (SEQ ID NO:11) with a peptide linker between T20 and Fc; T20 is shown in bold and the linker is italicized.

FIG. 4C shows the nucleic acid sequence of an Fc-T20-GS16 (SEQ ID NO:12) construct with a peptide linker between T20 and Fc; T20 is shown in bold and the linker is italicized.

FIG. 4D shows the nucleic acid sequence of a T20-Fc-GS18 (SEQ ID NO:13) construct with a peptide linker between T20 and Fc; T20 is shown in bold and the linker is italicized.

FIG. 4E shows the nucleic acid sequence of an Fc-T20-Phe-Cys (SEQ ID NO:14) construct with a peptide linker between T20 and Fc; T20 is shown in bold and the linker is italicized.

FIG. 4F shows the nucleic acid sequence of an Fc-T20 (SEQ ID NO:15) construct with a peptide linker between T20 and Fc and a His tag and an enterokinase sitelinked to the N terminus of Fc; T20 is shown in bold and the linker is italicized, the His tag is underlined and the enterokinase site is underlined and italicized.

FIG. 5 is a diagram representing the constructs for chimeric proteins with positioned linkers.

FIG. 6A demonstrates chemical conjugation of T20-Fc and T20-PEG-CysFc.

FIG. 6B demonstrates chemical conjugation of Fc-PEG-T20.

FIG. 7 is a graph demonstrating binding of Fc-T20 to soluble human Fc neonatal receptor (shFcRn).

FIG. 8A is a graph depicting pharmacokinetics of recombinantly produced Fc-T20 administered intravenously to monkeys.

FIG. 8B is a graph depicting pharmacokinetics of chemically conjugated Fc-T20 administered intravenously to monkeys.

FIG. 9 is a graph depicting the pharmacokinetics of recombinantly produced Fc-T20 administered orally to neonatal rats.

FIG. 10 demonstrates pharmacokinetics of Biotin-Fc-T20 administered to monkeys via the pulmonary route.

FIG. 11 is a diagram demonstrating reduced binding of Fc-T20 and T20-Fc to human serum albumin.

FIGS. 12A and B compares antiviral activity of Fc-T20 chimeric proteins to known HIV anti-viral agents.

FIG. 13A is the amino acid sequence of Fc-MESNA.

FIG. 13B is the nucleic acid sequence of Fc-MESNA.

### DESCRIPTION OF THE EMBODIMENTS

A. Definitions

Affinity tag, as used herein, means a molecule attached to a second molecule of interest, capable of interacting with a specific binding partner for the purpose of isolating or identifying said second molecule of interest.

Analogs of, or proteins or peptides substantially identical to the chimeric proteins of the invention, as used herein, means that a relevant amino acid sequence of a protein or a peptide is at least 70%, 75%, 80%, 85%, 90%, 95%, 97%, 98%, 99%, or 100% identical to, a given sequence. By way of example, such sequences may be variants derived from various species, or they may be derived from the given sequence by truncation, deletion, amino acid substitution or addition. Percent identity between two amino acid sequences is determined by standard alignment algorithms such as, for example, Basic Local Alignment Tool (BLAST) described in Altschul et al. 1990, J. Mol. Biol. 215:403-410, the algorithm of Needleman et al. 1970, J. Mol. Biol. 48:444-453; the algorithm of Meyers et al. 1988, Comput. Appl. Biosci. 4:11-17; or Tatusova et al. 1999, FEMS Microbiol. Lett. 174:247-250, etc. Such algorithms are incorporated into the BLASTN, BLASTP and “BLAST 2 Sequences” programs (see www.ncbi.nlm.nih.gov/BLAST). When utilizing such programs, the default parameters can be used. For example, for nucleotide sequences the following settings can be used for “BLAST 2 Sequences”: program BLASTN, reward for match 2, penalty for mismatch −2, open gap and extension gap penalties 5 and 2 respectively, gap x_dropoff 50, expect 10, word size 11, filter ON. For amino acid sequences the following settings can be used for “BLAST 2 Sequences”: program BLASTP, matrix BLOSUM62, open gap and extension gap penalties 11 and 1 respectively, gap x_dropoff 50, expect 10, word size 3, filter ON.

Bioavailability, as used herein, means the extent and rate at which a substance is absorbed into a living system or is made available at the site of physiological activity.

A chimeric protein, as used herein, refers to any protein comprised of a first amino acid sequence derived from a first source, bonded, covalently or non-covalently, to a second amino acid sequence derived from a second source, wherein the first and second source are not the same. A first source and a second source that are not the same can include two different biological entities, or two different proteins from the same biological entity, or a biological entity and a non-biological entity. A biological source can include any non-synthetically produced nucleic acid or amino acid sequence (e.g., a genomic or cDNA sequence, a plasmid or viral vector, a native virion or a mutant or analog, as further described herein, of any of the above). A synthetic source can include a protein or nucleic acid sequence produced chemically and not by a biological system (e.g., solid phase synthesis of amino acid sequences or nucleic acid sequences). A chimeric protein can also include a protein derived from at least 2 different synthetic sources or a protein derived from at least one biological source and at least one synthetic source.

DNA construct, as used herein, means a DNA molecule, or a clone of such a molecule, either single- or double-stranded that has been modified through human intervention to contain segments of DNA combined in a manner that as a whole would not otherwise exist in nature. DNA constructs contain the information necessary to direct the expression of polypeptides of interest. DNA constructs can include promoters, enhancers and transcription terminators. DNA constructs containing the information necessary to direct the secretion of a polypeptide will also contain at least one secretory signal sequence.

A fragment, as used herein, refers to a polypeptide comprising an amino acid sequence of at least 2 contiguous amino acid residues, of at least 5 contiguous amino acid residues, of at least 10 contiguous amino acid residues, of at least 15 contiguous amino acid residues, of at least 20 contiguous amino acid residues, of at least 25 contiguous amino acid residues, of at least 40 contiguous amino acid residues, of at least 50 contiguous amino acid residues, of at least 100 contiguous amino acid residues, or of at least 200 contiguous amino acid residues.

Linked, as used herein, refers to a first nucleic acid sequence covalently joined to a second nucleic acid sequence. The first nucleic acid sequence can be directly joined or juxtaposed to the second nucleic acid sequence or alternatively an intervening sequence can covalently join the first sequence to the second sequence. Linked as used herein can also refer to a first amino acid sequence covalently joined to a second amino acid sequence. The first amino acid sequence can be directly joined or juxtaposed to the second amino acid sequence or alternatively an intervening sequence can covalently join the first amino acid sequence to the second amino acid sequence. Linked as used herein can also refer to a first amino acid sequence covalently joined to a nucleic acid sequence or a small organic or inorganic molecule.

Operatively linked, as used herein, means a first nucleic acid sequence linked to a second nucleic acid sequence such that both sequences are capable of being expressed as a biologically active protein or peptide.

A small inorganic molecule, as used herein, means a molecule containing no carbon atoms and being no larger than 50 kD.

A small organic molecule, as used herein, means a molecule containing at least one carbon atom and being no larger than 50 kD.

High stringency, as used herein, includes conditions readily determined by the skilled artisan based on, for example, the length of the DNA. Generally, such conditions are defined as hybridization conditions as above, and with washing at approximately 68° C., 0.2×SSC, 0.1% SDS. The skilled artisan will recognize that the temperature and wash solution salt concentration can be adjusted as necessary according to factors such as the length of the probe.

Moderate stringency, as used herein, include conditions that can be readily determined by those having ordinary skill in the art based on, for example, the length of the DNA. The basic conditions are set forth by Sambrook et al. 1989, Molecular Cloning: A Laboratory Manual, 2d ed., 1:1.101-104, Cold Spring Harbor Laboratory Press, and include use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, such as Stark's solution, in 50% formamide at 42° C.), and washing conditions of 60° C., 0.5×SSC, 0.1% SDS.

Treat, treatment, treating, as used herein means, any of the following: the reduction in severity of a viral infection; the reduction in the duration of a disease course of a viral infection; the amelioration of one or more symptoms associated with a viral infection; the reduction of viral replication associated with a viral infection; the reduction of viral load or viral burden associated with a viral infection; the provision of beneficial effects to a subject with a viral infection, without necessarily curing the viral infection.

Viral fusion inhibitor, as used herein, means any molecule that prevents or decreases viral entry into a target cell by preventing or decreasing the close apposition of the virion surface with a membrane of the target cell.

B. Improvements Offered by Certain Embodiments of the Invention

The invention relates generally to improved viral fusion inhibitors. The invention relates to chimeric proteins wherein said chimeric proteins comprise at least one viral fusion inhibitor and at least a portion of an immunoglobulin constant region. The chimeric proteins of the invention provide improved chimeric proteins with viral fusion inhibitory activity compared to known peptide viral fusion inhibitors by providing for anti viral therapeutics with increased serum half life, increased bioavailabilty, increased stability and decreased binding to serum albumin. The chimeric proteins of the invention thus may require lower dosages and less frequent dosages to achieve suppression of, or inhibition of, viral entry into susceptible cells and thus decreased viral burden in an infected subject. Additionally, the invention provides for improved methods for administering viral fusion inhibitors. While viral fusion inhibitors are generally administered subcutaneously, intramuscularly or intravenously, the chimeric proteins of the invention can be administered using less invasive means such as oral, buccal, sublingual, nasal, ocular, administration, or pulmonary administration.

The invention also relates generally to improved methods of making chimeric proteins with viral fusion inhibitor activity. The invention relates to recombinant methods of producing chimeric proteins with viral fusion inhibitor activity. The invention thus relates to methods of making chimeric proteins wherein said chimeric proteins comprise at least one viral fusion inhibitor and at least a portion of an immunoglobulin constant region, said method comprising transfecting a cell with a DNA construct, said construct comprising a DNA sequence encoding at least a portion of an immunoglobulin constant region and a DNA sequence encoding at least one viral fusion inhibitor; culturing said cell under conditions such that the chimeric protein is expressed by said cell; and isolating said chimeric protein. The recombinant method of making the chimeric proteins of the invention are improved compared to known methods of making peptide viral fusion inhibitors. The methods of the invention provide higher product yields that are produced faster and at less cost than the chemical synthesis methods used to produce known peptide fusion inhibitors.

C. Chimeric Proteins

The invention relates to chimeric proteins comprising at least one viral fusion inhibitor, at least a portion of an immunoglobulin constant region, and optionally at least one linker. The portion of an immunoglobulin constant region will have both an N, or an amino terminus, and a C, or carboxy terminus. The chimeric protein may have the viral fusion inhibitor linked to the N terminus of the portion of an immunoglobulin constant region. Alternatively, the viral fusion inhibitor may be linked to the C terminus of the portion of an immunoglobulin constant region. In one embodiment, the linkage is a covalent bond. In another embodiment, the linkage is a non-covalent bond.

The chimeric protein can optionally comprise at least one linker; thus, the viral fusion inhibitor does not have to be directly linked to the portion of an immunoglobulin constant region. The linker can intervene in between the viral fusion inhibitor and the portion of an immunoglobulin constant region. The linker can be linked to the N terminus of the portion of an immunoglobulin constant region, or the C terminus of a the portion of an immunoglobulin constant region. If the viral fusion inhibitor is a polypeptide the viral fusion inhibitor will have an N terminus and a C terminus and the linker can be linked to the N terminus of the viral fusion inhibitor, or the C terminus the viral fusion inhibitor.

The invention thus relates to a chimeric protein comprised of at least one HIV fusion inhibitor (I), at least one linker (L) and at least a portion of an immunoglobulin constant region (F). In one embodiment, the invention relates to a chimeric protein comprised of the formula
I-L-F
wherein I is linked at its C terminus to the N terminus of L, and L is linked at its C terminus to the N terminus of F.

In another embodiment, the invention relates to a chimeric protein comprised of the formula
F-L-I
wherein F is linked at its C terminus to the N terminus of L, and L is linked at its C terminus to the N terminus of I.

In another embodiment, the invention relates to a chimeric protein comprised of the formula
A-F-L-I
wherein A is a first linker or an affinity tag, and F, L, and I are as described above and, wherein A is linked at its C terminus to the N terminus of F, and F is linked at its C terminus to the N terminus of L and L is linked at its C terminus to the N terminus of I. In one embodiment, A and L are not the same. In another embodiment, A and L are the same.

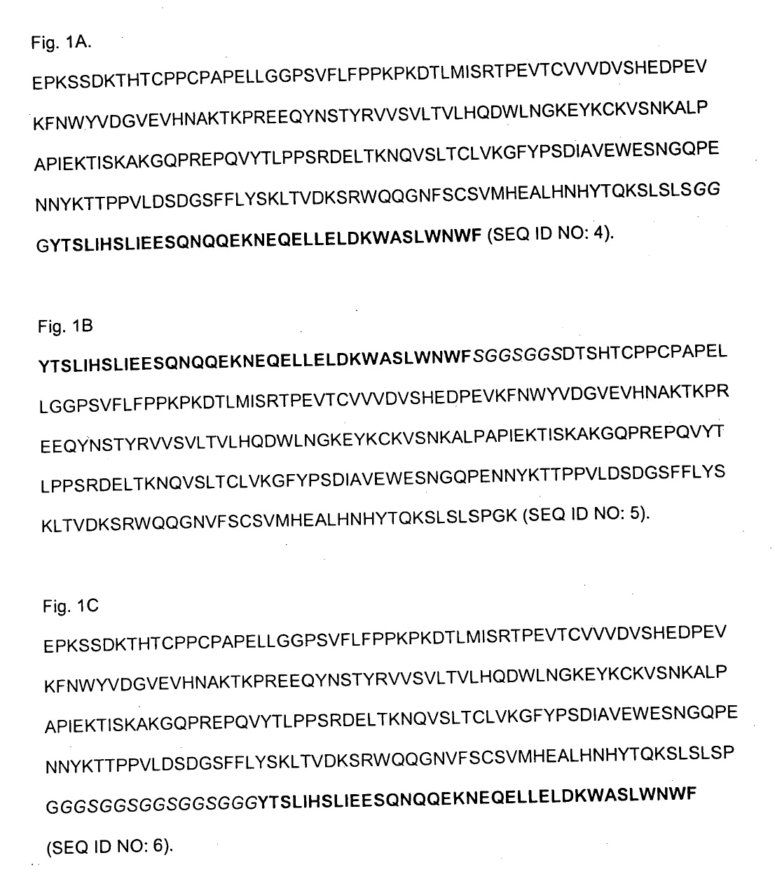

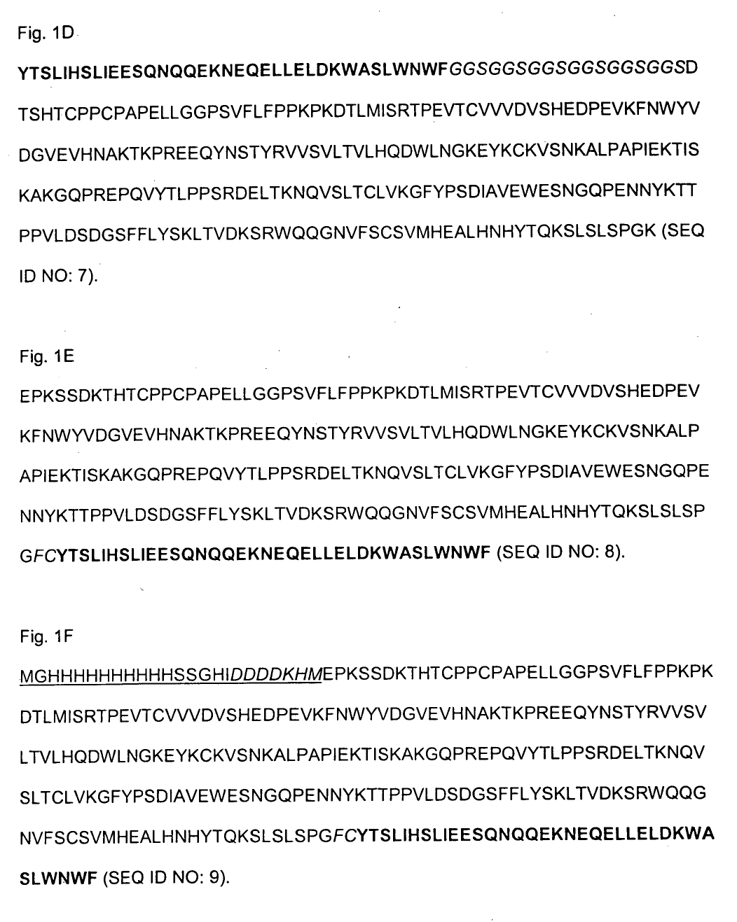

In one embodiment, the invention relates to a chimeric protein comprising the amino acid sequence of FIG. 1A (SEQ ID NO:4) or an analog thereof. In another embodiment, the invention relates to a chimeric protein comprising the amino acid sequence of FIG. 1B (SEQ ID NO:5) or an analog thereof. In another embodiment, the invention relates to a chimeric protein comprising the amino acid sequence of FIG. 1C (SEQ ID NO:6) or an analog thereof. In another embodiment, the invention relates to a chimeric protein comprising the amino acid sequence of FIG. 1D (SEQ ID NO:7) or an analog thereof. In another embodiment, the invention relates to a chimeric protein comprising the amino acid sequence of FIG. 1E (SEQ ID NO:8) or an analog thereof. In another embodiment, the invention relates to a chimeric protein comprising the amino acid sequence of FIG. 1F (SEQ ID NO:9) or an analog thereof.

The chimeric protein of the invention includes monomers, dimers, as well higher order multimers. In one embodiment, the chimeric protein is a monomer comprising one viral fusion inhibitor and one portion of an immunoglobulin constant region. In another embodiment, the chimeric protein is a dimer comprising two viral fusion inhibitors and two portions of an immunoglobulin. In one embodiment, the two viral fusion inhibitors are the same. In one embodiment, the two viral fusion inhibitors are different. In one embodiment, the two portions of an immunoglobulin are the same. In one embodiment, the two portions of an immunoglobulin are different. In one embodiment, the two viral fusion inhibitors are different. In another embodiment, the chimeric protein is a monomer/dimer hybrid comprising a first chain and a second chain, wherein said first chain comprises at least a portion of an immunoglobulin constant region linked to a viral fusion inhibitor and said second chain comprises at least a portion of an immunoglobulin constant region without a viral fusion inhibitor linked to it. Such chimeric proteins may be described using the formulas set forth in Table 1, where I, L, and F are as described above, and where (′) indicates a different molecule than without (′) and where (:) indicates at least one non-peptide bond.

TABLE 1

I-F:F-I
I′-F:F-I
I-L-F:F-I
I-L-F:F-L-I
I′-L-F:F-L-I
I-L′-F:F-L-I
I′-L′-F:F-L-I
F:F-I
F:F-L-I
I-F:F
I-L-F:F
L-F:F-I

The skilled artisan will understand additional combinations are possible including the use of additional linkers.

1. Chimeric Protein Variants

Derivatives of the chimeric proteins of the invention, antibodies against the chimeric proteins of the invention and antibodies against binding partners of the chimeric proteins of the invention are all contemplated, and can be made by altering their amino acids sequences by substitutions, additions, and/or deletions/truncations or by introducing chemical modification that result in functionally equivalent molecules. It will be understood by one of ordinary skill in the art that certain amino acids in a sequence of any protein may be substituted for other amino acids without adversely affecting the activity of the protein.

Various changes may be made in the amino acid sequences of the the chimeric proteins of the invention or DNA sequences encoding therefore without appreciable loss of their biological activity, function, or utility. Derivatives, analogs, or mutants resulting from such changes and the use of such derivatives is within the scope of the present invention. In a specific embodiment, the derivative is functionally active, i.e., capable of exhibiting one or more activities associated with the chimeric proteins of the invention, e.g., inhibiting viral fusion. Many assays capable of testing the activity of a chimeric protein comprising a viral fusion inhibitor are known in the art. Where the viral fusion inhibitor is an HIV viral fusion inhibitor activity can be tested by measuring reverse transcriptase activity using known methods (see, e.g., Barre-Sinoussi et al. 1983, Science 220:868; Gallo et al. 1984, Science 224:500). Alternatively activity can be measured by measuring fusogenic activity directly (see, e.g., Nussbaum et al. 1994, J. Virol. 68(9):5411).

Substitutes for an amino acid within the sequence may be selected from other members of the class to which the amino acid belongs (see Table 2). Furthermore, various amino acids are commonly substituted with neutral amino acids, e.g., alanine, leucine, isoleucine, valine, proline, phenylalanine, tryptophan, and methionine (see, e.g., MacLennan et al. 1998, Acta Physiol. Scand. Suppl. 643:55-67; Sasaki et al. 1998, Adv. Biophys. 35:1-24).

TABLE 2

| Original | Exemplary | Typical |
|---|---|---|
| Residues | Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, | Ile |
| | Ala, Phe | |
| Lys (K) | Arg, 1,4-Diamino-butyric | Arg |
| | Acid, Gln, Asn | |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, | Leu |
| | Norleucine | |

2. Viral Fusion Inhibitors

The viral fusion inhibitor for use in the chimeric protein can be any molecule which decreases or prevents viral penetration of a cellular membrane of a target cell. The viral fusion inhibitor can be any molecule that decreases or prevents the formation of syncytia between at least two susceptible cells. The viral fusion inhibitor can be any molecule which decreases or prevents the joining of, a lipid bilayer membrane of a eukaryotic cell, and a lipid bilayer of an enveloped virus. Examples of enveloped virus include, but are not limited to HIV-1, HIV-2, SIV, influenza, parainfluenza, Epstein-Barr virus, CMV, herpes simplex 1, herpes simplex 2 and respiratory syncytia virus.

The viral fusion inhibitor can be any molecule that decreases or prevents viral fusion including, but not limited to, a protein, a protein fragment, a peptide, a peptide fragment, a small organic molecule or a small inorganic molecule (see, e.g., U.S. Pat. Nos. 6,086,875, 6,030,613, 6,485,726, PCT Application No. US/02/21335). In one embodiment the fusion inhibitor is a peptide. In one embodiment, the viral fusion inhibitor is a peptide of 3-36 amino acids. In another embodiment, the viral fusion inhibitor is a polypeptide of 3-50 amino acids, 10-65 amino acids, 10-75 amino acids. The peptide can be comprised of a naturally occurring amino acid sequence (e.g. a fragment of gp41) including analogs and mutants thereof or the peptide can be comprised of an amino acid sequence not found in nature, so long as the peptide exhibits viral fusion inhibitory activity.

In one embodiment, the viral fusion inhibitor is a protein, a protein fragment, a peptide, a peptide fragment identified as being a viral fusion inhibitor using at least one computer algorithm, e.g., ALLMOTI5, 107×178×4 and PLZIP (see, e.g., U.S. Pat. Nos. 6,013,263; 6,015,881; 6,017,536; 6,020,459; 6,060,065; 6,068,973; 6,093,799; and 6,228,983).

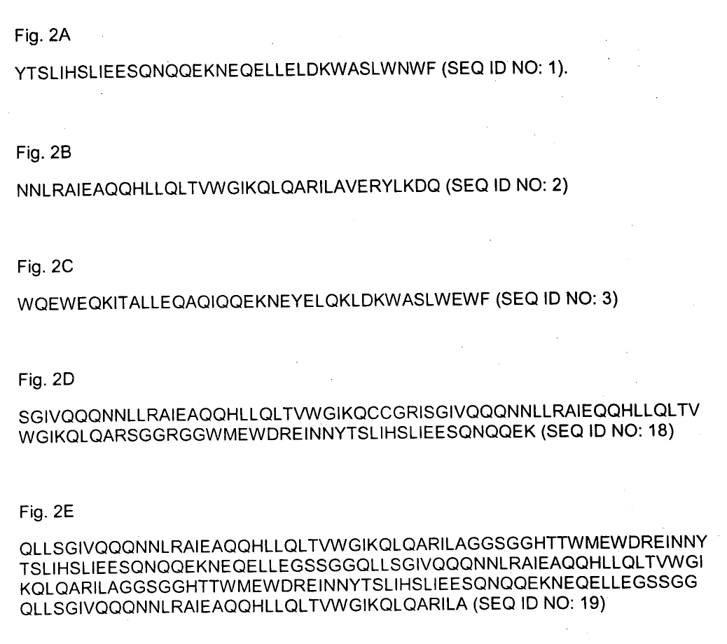

In one embodiment, the viral fusion inhibitor is an HIV fusion inhibitor. In one embodiment, HIV is HIV-1. In another embodiment, HIV is HIV-2. In one embodiment the HIV fusion inhibitor is a peptide comprised of a fragment of the gp41 envelope protein of HIV-1. The HIV fusion inhibitor can comprise, e.g., T20 (FIG. 2A)(SEQ ID NO:1) or an analog thereof, T21 (FIG. 2B) (SEQ ID NO:2) or an analog thereof, T1249 (FIG. 2C)(SEQ ID NO:3) or an analog thereof, NCCGgp41 (Louis et al. 2001, J. Biol. Chem. 276:(31)29485) (FIG. 2D) (SEQ ID NO:17) or an analog thereof, or 5 helix (Root et al. 2001, Science 291:884) (FIG. 2E)(SEQ ID NO:18) or an analog thereof.

Assays known in the art can be used to test for viral fusion inhibiting activity of a polypeptide, a small organic molecule, or a small inorganic molecule. These assays include a reverse transcriptase assay, a p24 assay, or syncytia formation assay (see, e.g., U.S. Pat. No. 9,464,933).

3. Immunoglobulins

The chimeric proteins of the invention comprise at least a portion of an immunoglobulin constant region. Immunoglobulins are comprised of four protein chains that associate covalently-two heavy chains and two light chains. Each chain is further comprised of one variable region and one constant region. Depending upon the immunoglobulin isotype, the heavy chain constant region is comprised of 3 or 4 constant region domains (e.g. CH1, CH2, CH3, CH4). Some isotypes are further comprised of a hinge region.

The portion of an immunoglobulin constant region can be a portion of an immunoglobulin constant region obtained from any mammal. The portion of an immunoglobulin constant region can include a portion of a human immunoglobulin, a non-human primate immunoglobulin, a bovine immunoglobulin, a porcine immunoglobulin, a murine immunoglobulin, an ovine immunoglobulin or a rat immunoglobulin.

The immunoglobulin can be produced recombinantly or synthetically. The immunoglobulin can be isolated from a cDNA library. The immunoglobulin can be isolated from a phage library (see, e.g., McCafferty et al. 1990, Nature 348: 552). The immunoglobulin can be obtained by gene shuffling of known sequences (Mark et al. 1992, Bio/Technol. 10: 779). The immunoglobulin can be isolated by in vivo recombination (Waterhouse et al. 1993, Nucl. Acid Res. 21: 2265). The immunoglobulin can be a humanized immunoglobulin (Jones et al. 1986, Nature 332: 323).

The portion of an immunoglobulin constant region can include a portion of an IgG, an IgA, an IgM, an IgD, an IgE. In one embodiment, the immunoglobulin is an IgG. In another embodiment, the immunoglobulin is IgG1.

The portion of an immunoglobulin constant region can include the entire heavy chain constant region, or a fragment or analog thereof. A heavy chain constant region can comprise a CH1 domain, a CH2 domain, a CH3 domain, and/or a hinge region. A constant region can comprise a CH1 domain, a CH2 domain, a CH3 domain, and/or a CH4 domain.

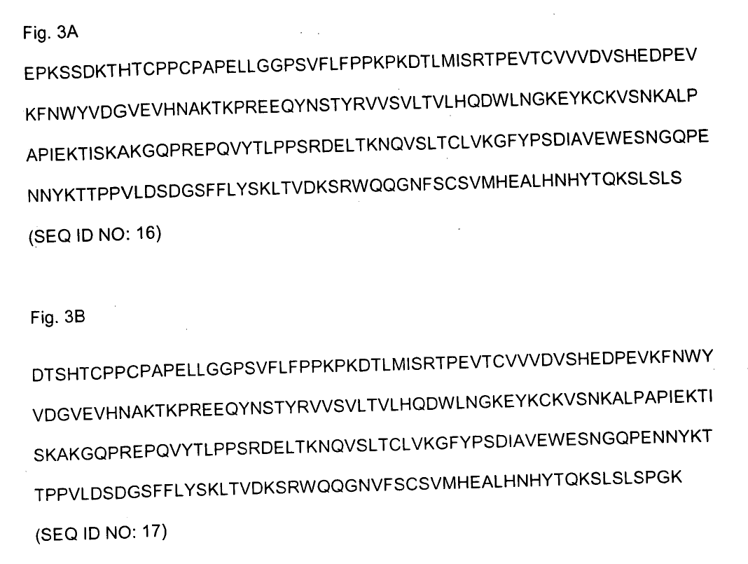

The portion of an immunoglobulin constant region can include an Fc fragment. An Fc fragment can be comprised of the CH2 and CH3 domains of an immunoglobulin and the hinge region of the immunoglobulin. The Fc fragment can be the Fc fragment of an IgG1, an IgG2, an IgG3 or an IgG4. In one embodiment, the portion of an immunoglobulin constant region is an Fc fragment of an IgG1. In another embodiment, the portion of an immunoglobulin constant region is comprised of SEQ ID NO:16 (FIG. 3A) or an analog thereof. In another embodiment, the portion of an immunoglobulin constant region is comprised of SEQ ID NO:17 (FIG. 3B) or an analog thereof.

The portion of an immunoglobulin constant region can include an Fc variant. Fc variant refers to a molecule or sequence that is modified from a native Fc but still comprises a binding site for the salvage receptor, FcRn (WO 97/34631). Native refers to an Fc that has not been modified by a human. WO 96/32478 describe exemplary Fc variants, as well as interaction with the salvage receptor. Thus, the term “Fc variant” comprises a molecule or sequence that is humanized from a non-human native Fc. Furthermore, a native Fc comprises sites that may be removed because they provide structural features or biological activity that are not required for the fusion molecules of the present invention. Thus, Fc variant comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

In another embodiment, the portion of an immunoglobulin constant region is a neonatal Fc receptor (FcRn) binding partner. An FcRn binding partner is any molecule that can be specifically bound by the FcRn receptor with consequent active transport by the FcRn receptor of the FcRn binding partner. The FcRn receptor has been isolated from several mammalian species including humans. The sequences of the human FcRn, rat FcRn, and mouse FcRn are known (Story et al. 1994, J. Exp. Med. 180:2377). The FcRn receptor binds IgG (but not other immunoglobulin classes such as IgA, IgM, IgD, and IgE) at relatively low pH, actively transports the IgG transcellularly in a luminal to serosal direction, and then releases the IgG at relatively higher pH found in the interstitial fluids. One result of the active transport is to increase the serum half-life of FcRn binding partners, including chimeric proteins comprised of FcRn binding partners. It is expressed in adult epithelial tissue (U.S. Pat. Nos. 6,030,613 and 6,086,875) including lung and intestinal epithelium (Israel et al. 1997, Immunology 92:69) renal proximal tubular epithelium (Kobayashi et al. 2002, Am. J. Physiol. Renal Physiol. 282:F358) as well as nasal epithelium, vaginal surfaces, and biliary tree surfaces.

FcRn binding partners of the present invention encompass any molecule that can be specifically bound by the FcRn receptor including whole IgG, the Fc fragment of IgG, and other fragments that include the complete binding region of the FcRn receptor. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The major contact area of the Fc with the FcRn is near the junction of the CH2 and CH3 domains. Fc-FcRn contacts are all within a single Ig heavy chain. The FcRn binding partners include whole IgG, the Fc fragment of IgG, and other fragments of IgG that include the complete binding region of FcRn. The major contact sites include amino acid residues 248, 250-257, 272, 285, 288, 290-291, 308-311, and 314 of the CH2 domain and amino acid residues 385-387, 428, and 433-436 of the CH3 domain. References made to amino acid numbering of immunoglobulins or immunoglobulin fragments, or regions, are all based on Kabat et al. 1991, Sequences of Proteins of Immunological Interest, U.S. Department of Public Health, Bethesda, Md.

The skilled artisan will understand that portions of an immunoglobulin constant region for use in the chimeric protein of the invention can include mutants or analogs thereof, or can include chemically modified immunoglobulin constant regions (e.g., pegylation) (see, e.g., Aslam and Dent 1998, Bioconjugation: Protein Coupling Techniques For the Biomedical Sciences Macmilan Reference, London) or fragments thereof. In one instance a mutant can provide for enhanced binding of an FcRn binding partner for the FcRn. Also contemplated for use in the chimeric protein of the invention are peptide mimetics of at least a portion of an immunoglobulin constant region, e.g., a peptide mimetic of an Fc fragment or a peptide mimetic of an FcRn binding partner. In one embodiment, the peptide mimetic is identified using phage display (see, e.g., McCafferty et al. 1990, Nature 348:552, Kang et al. 1991, Proc. Natl. Acad. Sci. USA 88:4363; EP 0 589 877 B1).

The Fc region of IgG can be modified according to well recognized procedures such as site directed mutagenesis and the like to yield modified IgG or Fc fragments or portions thereof that will be bound by FcRn. Such modifications include modifications remote from the FcRn contact sites as well as modifications within the contact sites that preserve or even enhance binding to the FcRn. For example the following single amino acid residues in human IgG1 Fc (Fcγ1) can be substituted without significant loss of Fc binding affinity for FcRn: P238A, S239A, K246A, K248A, D249A, M252A, T256A, E258A, T260A, D265A, S267A, H268A, E269A, D270A, E272A, L274A, N276A, Y278A, D280A, V282A, E283A, H285A, N286A, T289A, K290A, R292A, E293A, E294A, Q295A, Y296F, N297A, S298A, Y300F, R301A, V303A, V305A, T307A, L309A, Q311A, D312A, N315A, K317A, E318A, K320A, K322A, S324A, K326A, A327Q, P329A, A330Q, A330S, P331A, P331S, E333A, K334A, T335A, S337A, K338A, K340A, Q342A, R344A, E345A, Q347A, R355A, E356A, M358A, T359A, K360A, N361A, Q362A, Y373A, S375A D376A, A378Q, E380A, E382A, S383A, N384A, Q386A, E388A, N389A, N390A, Y391F, K392A, L398A, S400A, D401A, D413A, K414A, R416A, Q418A, Q419A, N421A, V422A, S424A, E430A, N434A, T437A, Q438A, K439A, S440A, S444A, and K447A, where for example P238A represents wildtype proline substituted by alanine at position number 238. In addition to alanine other amino acids may be substituted for the wildtype amino acids at the positions specified above. Mutations may be introduced singly into Fc giving rise to more than one hundred FcRn binding partners distinct from native Fc. Additionally, combinations of two, three, or more of these individual mutations may be introduced together, giving rise to hundreds more FcRn binding partners.

Certain of the above mutations may confer new functionality upon the FcRn binding partner. For example, one embodiment incorporates N297A, removing a highly conserved N-glycosylation site. The effect of this mutation is to reduce immunogenicity, thereby enhancing circulating half life of the FcRn binding partner, and to render the FcRn binding partner incapable of binding to FcγRI, FcγRIIA, FcγRIIB, and FcγRIIIA, without compromising affinity for FcRn (Routledge et al. 1995, Transplantation 60:847; Friend et al. 1999, Transplantation 68:1632; Shields et al. 1995, J. Biol. Chem. 276:6591). Additionally, at least three human Fc gamma receptors appear to recognize a binding site on IgG within the lower hinge region, generally amino acids 234-237. Therefore, another example of new functionality and potential decreased immunogenicity may arise from mutations of this region, as for example by replacing amino acids 233-236 of human IgG1 “ELLG” to the corresponding sequence from IgG2 “PVA” (with one amino acid deletion). It has been shown that FcγRI, FcγRII, and FcγRIII, which mediate various effector functions will not bind to IgG1 when such mutations have been introduced. Ward and Ghetie 1995, Therapeutic Immunology 2:77 and Armour et al. 1999, Eur. J. Immunol. 29:2613. As a further example of new functionality arising from mutations described above affinity for FcRn may be increased beyond that of wild type in some instances. This increased affinity may reflect an increased “on” rate, a decreased “off” rate or both an increased “on” rate and a decreased “off” rate. Mutations believed to impart an increased affinity for FcRn include T256A, T307A, E380A, and N434A (Shields et al. 2001, J. Biol. Chem. 276:6591).

In one embodiment, the FcRn binding partner is a polypeptide including the sequence PKNSSMISNTP (SEQ ID NO: 22) and optionally further including a sequence selected from HQSLGTQ (SEQ ID NO: 23), HQNLSDGK (SEQ ID NO: 24), HQNISDGK (SEQ ID NO: 25), or VISSHLGQ (SEQ ID NO: 26)(U.S. Pat. No. 5,739,277).

Two FcRn receptors can bind a single Fc molecule. Crystallographic data suggest that each FcRn molecule binds a single polypeptide of the Fc dimer. Linking the FcRn binding partner, e.g., an Fc fragment of an IgG, to a viral fusion inhibitor thus provides a means of delivering the viral fusion inhibitor through any mucosal surface, e.g., orally or as an aerosol administered nasally or via a pulmonary route. Also contemplated are buccal administration, ocular administration, rectal administration and vaginal administration.

4. Optional Linkers

The chimeric protein of the invention can optionally comprise at least one linker molecule. The linker can be comprised of any organic molecule. In one embodiment the linker is polyethylene glycol (PEG). The PEG can be in the range of 0.1 kd to 20 kd. The linker can be a dendrimer allowing multiple species, e.g., small organic molecules or small inorganic molecules to be linked to at least a portion of an immunoglobulin constant region. In another embodiment, the linker is comprised of amino acids. The linker can comprise 1-5 amino acids, 1-10 amino acids, 1-20 amino acids, 10-50 amino acids, 50-100 amino acids, or 100-200 amino acids. The linker can comprise the sequence Gn. The linker can comprise the sequence (GGS)ₙ. In both instances, n may be an integer from 1 to 10, for example, 1, 2, 3, 4, 5, 6, 7, 8, 9, or 10. Examples of linkers include, but are not limited to, GGG (SEQ ID NO: 27), SGGSGGS (SEQ ID NO: 28), GGSGGSGGSGGSGGG, GGSGGSGGSGGSGGSGGS (SEQ ID NO: 29), and FC.

The linker may also incorporate a moiety capable of being cleaved either chemically (e.g. hydrolysis of an ester bond), enzymatically (i.e. incorporation of a protease cleavage sequence) or photolytically (e.g., a chromophore such as 3-amino-3-(2-nitrophenyl) proprionic acid (ANP)) in order to release the biologically active molecule from the Fc protein.

The linker does not eliminate the anti-fusogenic activity of the viral fusion inhibitor. Optionally, the linker enhances the anti-fusogenic activity of the viral fusion inhibitor, e.g., by diminishing the effects of steric hindrance and making the viral fusion inhibitor more accessible to its target binding site, e.g., a viral protein, gp41.

D. Nucleic Acid Constructs

The invention relates to a nucleic acid construct comprising a nucleic acid sequence encoding the chimeric proteins of the invention, said nucleic acid sequence comprising a first nucleic acid sequence encoding, for example, a viral fusion inhibitor, operatively linked to a second nucleic acid sequence encoding at least a portion of an immunoglobulin constant region. The nucleic acid sequence can also include additional sequences or elements known in the art (e.g. promoters, enhancers, poly A sequences). The nucleic acid sequence can optionally include a nucleic acid sequence encoding a linker placed between the nucleic acid sequence encoding the viral fusion inhibitor and the portion of the immunoglobulin. The nucleic acid sequence can optionally include a linker sequence placed before or after the nucleic acid sequence encoding the viral fusion inhibitor and the portion of the immunoglobulin. In one embodiment, the nucleic acid construct is comprised of DNA. In another embodiment, the nucleic acid construct is comprised of RNA. The nucleic acid construct can be a vector, e.g., a viral vector or a plasmid. Examples of viral vectors include, but are not limited to, adeno virus vector, an adeno associated virus vector, or a murine leukemia virus vector. Examples of plasmids include but are not limited to pUC and pGEX.

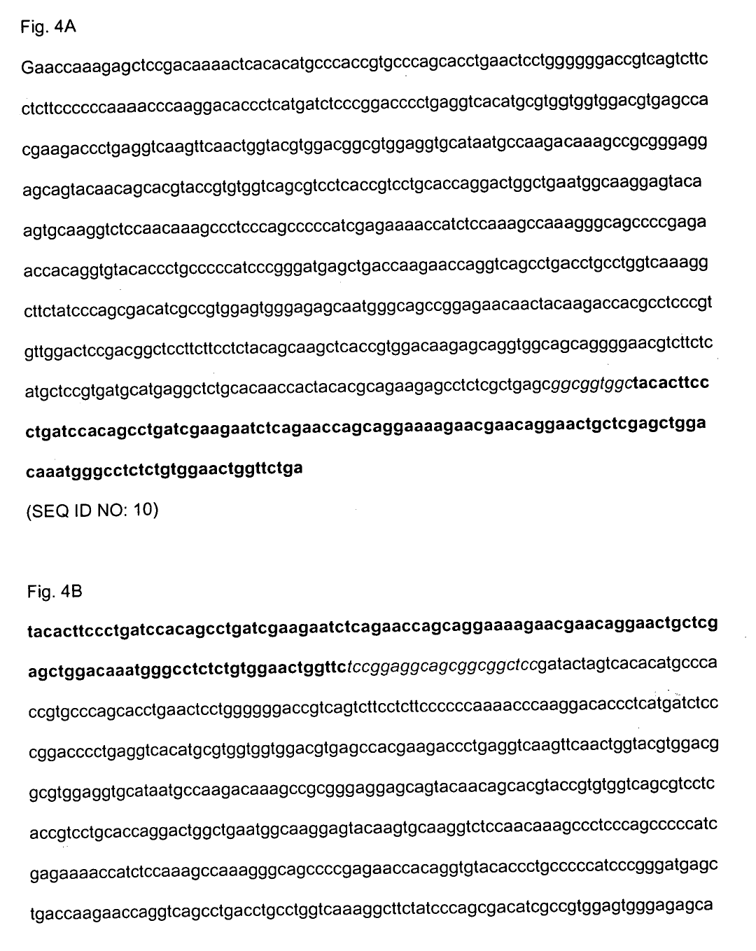

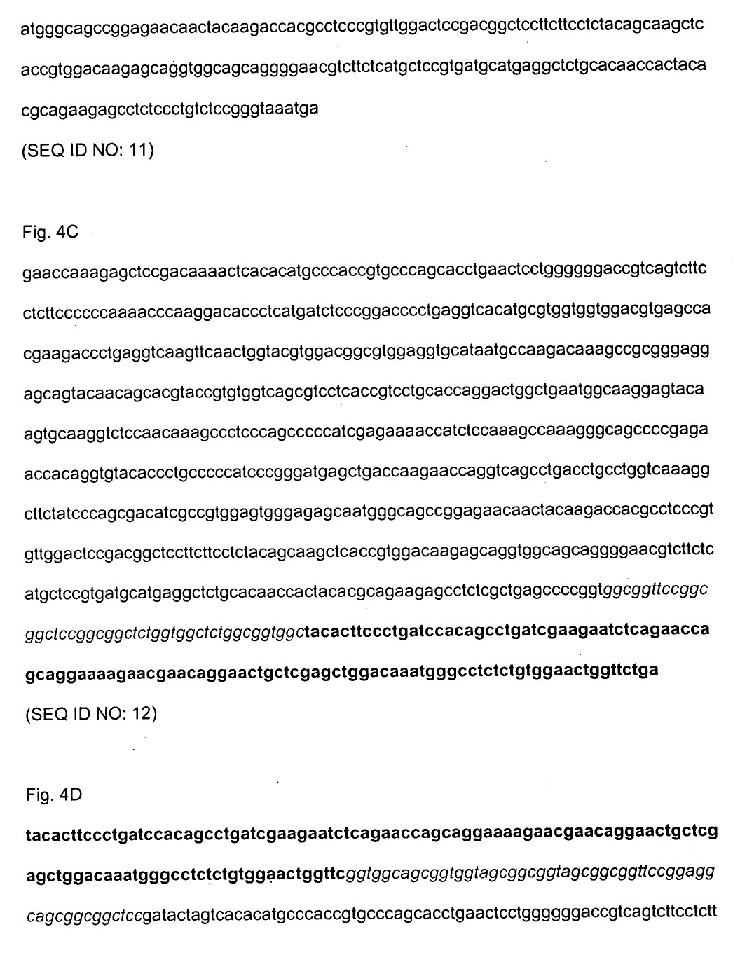

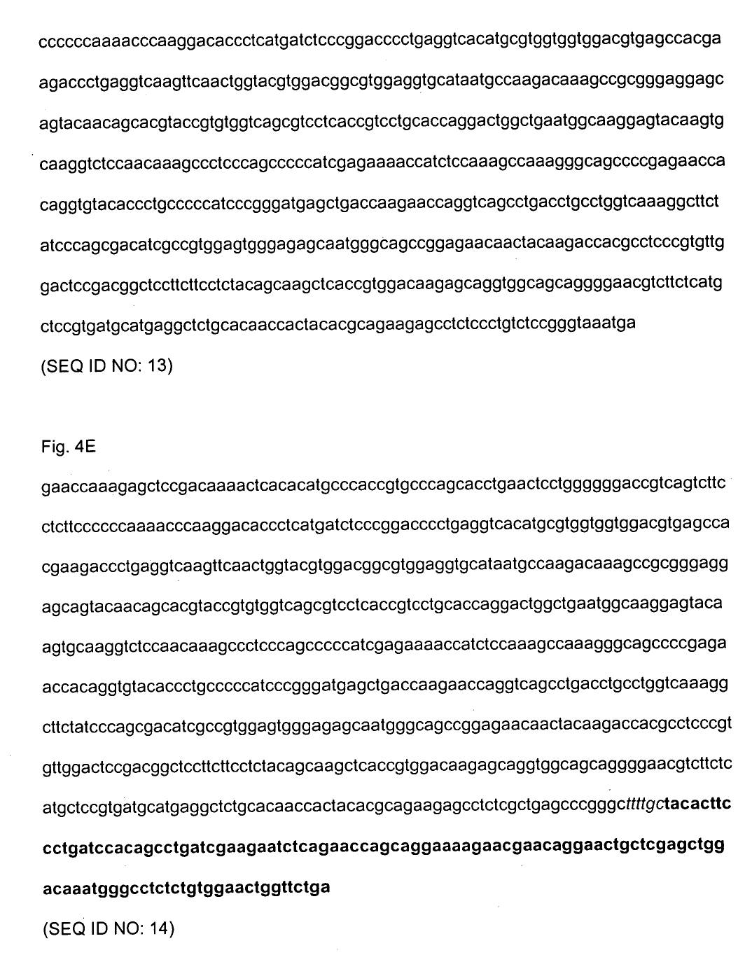

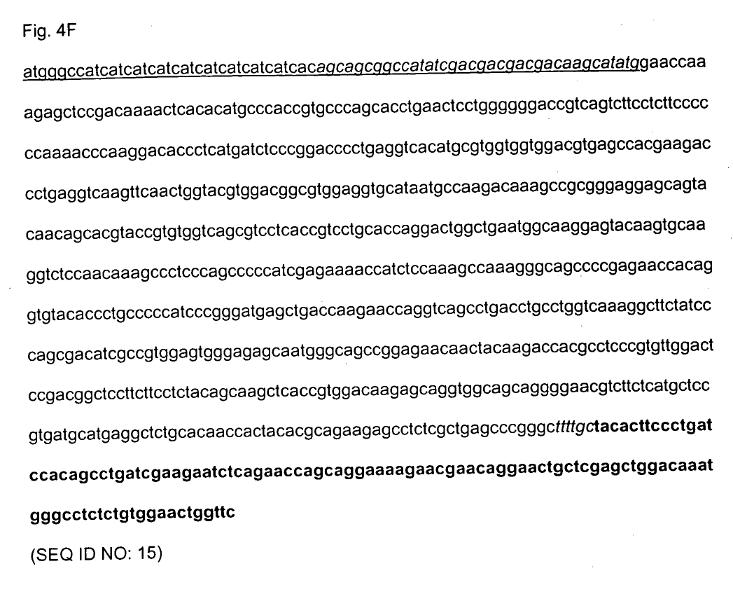

In one embodiment, the nucleic acid construct comprises the nucleic acid sequence of FIG. 4A (SEQ ID NO:10). In one embodiment, the nucleic acid construct comprises the nucleic acid sequence of FIG. 4B (SEQ ID NO:11). In one embodiment, the nucleic acid construct comprises the nucleic acid sequence of FIG. 4C (SEQ ID NO:12). In one embodiment, the nucleic acid construct comprises the nucleic acid sequence of FIG. 4D (SEQ ID NO:13). In one embodiment, the nucleic acid construct comprises the nucleic acid sequence of FIG. 4E (SEQ ID NO:14). In one embodiment, the nucleic acid construct comprises the nucleic acid sequence of FIG. 4F (SEQ ID NO:15).

Due to the known degeneracy of the genetic code, wherein more than one codon can encode the same amino acid, a DNA sequence can vary from that shown in SEQ ID NOS:10, 11, 12, 13, 14 or 15 and still encode a polypeptide having the corresponding amino acid sequence of SEQ ID NOS:4, 5, 6, 7, 8, or 9. Such variant DNA sequences can result from silent mutations (e.g., occurring during PCR amplification), or can be the product of deliberate mutagenesis of a native sequence. The invention thus provides isolated DNA sequences encoding polypeptides of the invention, selected from: (a) DNA comprising the nucleotide sequence of SEQ ID NOS:10, 11, 12, 13, 14 or 15; (b) DNA encoding the polypeptides of SEQ ID NOS:4, 5, 6, 7, 8, and 9; (c) DNA capable of hybridization to a DNA of (a) or (b) under conditions of moderate stringency and which encodes polypeptides of the invention; (d) DNA capable of hybridization to a DNA of (a) or (b) under conditions of high stringency and which encodes polypeptides of the invention, and (e) DNA which is degenerate as a result of the genetic code to a DNA defined in (a), (b), (c), or (d) and which encode polypeptides of the invention. Of course, polypeptides encoded by such DNA sequences are encompassed by the invention.

In another embodiment, the nucleic acid molecules of the invention also comprise nucleotide sequences that are at least 80% identical to a native sequence. Also contemplated are embodiments in which a nucleic acid molecule comprises a sequence that is at least 90% identical, at least 95% identical, at least 98% identical, at least 99% identical, or at least 99.9% identical to a native sequence. The percent identity may be determined by visual inspection and mathematical calculation. Alternatively, the percent identity of two nucleic acid sequences can be determined by comparing sequence information using the GAP computer program, version 6.0 described by Devereux et al. 1984, Nucl. Acids Res. 12:387 and available from the University of Wisconsin Genetics Computer Group (UWGCG). The preferred default parameters for the GAP program include: (1) a unary comparison matrix (containing a value of 1 for identities and 0 for non identities) for nucleotides, and the weighted comparison matrix of Gribskov and Burgess 1986, Nucl. Acids Res. 14:6745, as described by Schwartz and Dayhoff, eds. 1979, Atlas of Protein Sequence and Structure, National Biomedical Research Foundation, pp. 353-358 (2) a penalty of 3.0 for each gap and an additional 0.10 penalty for each symbol in each gap; and (3) no penalty for end gaps. Other programs used by one skilled in the art of sequence comparison may also be used.

E. Synthesis of Chimeric Proteins

Chimeric proteins comprising at least a portion of an immunoglobulin constant region and a viral fusion inhibitor can be synthesized using techniques well known in the art. For example, the chimeric proteins of the invention can be synthesized recombinantly in cells (see, e.g., Sambrook et al. 1989, Molecular Cloning A Laboratory Manual, Cold Spring Harbor Laboratory, N.Y. and Ausubel et al. 1989, Current Protocols in Molecular Biology, Greene Publishing Associates and Wiley Interscience, N.Y.). Alternatively, the chimeric proteins of the invention can be synthesized using known synthetic methods such as solid phase synthesis. Synthetic techniques are well known in the art (see, e.g., Merrifield 1973, Chemical Polypeptides, (Katsoyannis and Panayotis eds.) pp. 335-61; Merrifield 1963, J. Am. Chem. Soc. 85:2149; Davis et al. 1985, Biochem. Intl. 10:394; Finn et al. 1976, The Proteins (3^{rd} ed.) 2:105; Erikson et al. 1976, The Proteins (^{2nd} ed.) 2:257; U.S. Pat. No. 3,941,763. Alternatively, the chimeric proteins of the invention can be synthesized using a combination of recombinant and synthetic methods. In certain applications, it may be beneficial to use either a recombinant method or a combination of recombinant and synthetic methods.

Nucleic acids encoding peptide viral fusion inhibitors can be readily synthesized using recombinant techniques well known in the art. Alternatively, the peptides themselves can be chemically synthesized (see, e.g., U.S. Pat. Nos. 6,015,881; 6,281,331; 6,469,136).

DNA sequences encoding immunoglobulins or fragments thereof may be cloned from a variety of genomic or cDNA libraries known in the art. The techniques for isolating such DNA sequences using probe-based methods are conventional techniques and are well known to those skilled in the art. Probes for isolating such DNA sequences may be based on published DNA sequences (see, for example, Hieter et al. 1980, Cell 22: 197-207). The polymerase chain reaction (PCR) method disclosed by Mullis et al. (U.S. Pat. No. 4,683,195) and Mullis (U.S. Pat. No. 4,683,202) may be used. The choice of library and selection of probes for the isolation of such DNA sequences is within the level of ordinary skill in the art. Alternatively, DNA sequences encoding immunoglobulins or fragments thereof can be obtained from vectors known in the art to contain immunoglobulins or fragments thereof.

For recombinant production, a polynucleotide sequence encoding the chimeric protein is inserted into an appropriate expression vehicle, i.e., a vector which contains the necessary elements for the transcription and translation of the inserted coding sequence, or in the case of an RNA viral vector, the necessary elements for replication and translation. The nucleic acid encoding the chimeric protein is inserted into the vector in proper reading frame.

The expression vehicle is then transfected into a suitable target cell which will express the peptide. Transfection techniques known in the art include, but are not limited to, calcium phosphate precipitation (Wigler et al. 1978, Cell 14:725) and electroporation (Neumann et al. 1982, EMBO J. 1:841). A variety of host-expression vector systems may be utilized to express the chimeric proteins described herein including both prokaryotic (e.g. E. coli) or eukaryotic cells. These include, but are not limited to, microorganisms such as bacteria (e.g. E. coli) transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems, including mammalian cells (e.g., CHO, Cos, HeLa cells, myeloma cells). When the chimeric protein is expressed in a eukaryotic cell the DNA encoding the chimeric protein may also code for a signal sequence that will permit the chimeric protein to be secreted. One skilled in the art will understand that while the signal sequence is translated it is cleaved by the cell to form the mature chimeric protein. Various signal sequences are known in the art, e.g., interferon α signal sequence and the mouse Igκ light chain signal sequence. Alternatively, where a signal sequence is not included the chimeric protein can be recovered by lysing the cells.

When the chimeric protein of the invention is recombinantly synthesized in a prokaryotic cell it may be desirable to refold the protein. The chimeric protein produced by this method can be refolded to a biologically active conformation using conditions known in the art, e.g., denaturing and reducing conditions and then dialyzed slowly into PBS.

Depending on the expression system used, the expressed peptide is then isolated by procedures well-established in the art (e.g., affinity chromatography, size exclusion chromatography, ion exchange chromatography).

The expression vectors can encode for tags that permit for easy purification of the recombinantly produced protein. Examples include, but are not limited to, histidine tags, flag tags, maltose protein binding tags. In one example vector pUR278 (Ruther et al. 1983, EMBO J. 2:1791) may be used in which the chimeric protein described herein coding sequence may be ligated into the vector in frame with the lac z coding region so that a hybrid protein is produced. In another example PGEX vectors may be used to express proteins with a glutathione S-transferase (GST) tag. These proteins are usually soluble and can easily be purified from cells by adsorption to glutathione-agarose beads followed by elution in the presence of free glutathione. The vectors include cleavage sites (thrombin or factor Xa protease or PreScission Protease™ (Pharmacia, Peapack, N.J.) for easy removal of the tag after purification.

To increase efficiency of production, the polynucleotide can be designed to encode multiple units of the chimeric protein of the invention separated by enzymatic cleavage sites. The resulting polypeptide can be cleaved (e.g. by treatment with the appropriate enzyme) in order to recover the peptide units. This can increase the yield of peptides driven by a single promoter. When used in appropriate viral expression systems, the translation of each peptide encoded by the mRNA is directed internally in the transcript, e.g., by an internal ribosome entry site, IRES. Thus, the polycistronic construct directs the transcription of a single, large polycistronic mRNA which, in turn, directs the translation of multiple, individual peptides. This approach eliminates the production and enzymatic processing of polyproteins and may significantly increase yield of peptide driven by a single promoter.

Vectors used in transformation will usually contain a selectable marker used to identify transformants. In bacterial systems this can include an antibiotic resistance gene such as ampicillin or kanamycin. Selectable markers for use in cultured mammalian cells include genes that confer resistance to drugs, such as neomycin, hygromycin, and methotrexate. The selectable marker may be an amplifiable selectable marker. One amplifiable selectable marker is the DHFR gene. Another amplifiable marker is the DHFRr cDNA (Simonsen and Levinson 1983, Proc. Natl. Acad. Sci. (USA) 80:2495). Selectable markers are reviewed by Thilly (Mammalian Cell Technology, Butterworth Publishers, Stoneham, Mass.) and the choice of selectable markers is well within the level of ordinary skill in the art.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339).

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the ³⁵S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter; the CMV promoter) may be used; when generating cell lines that contain multiple copies of expression product, SV40-, BPV- and EBV-based vectors may be used with an appropriate selectable marker.

In cases where plant expression vectors are used, the expression of sequences encoding linear or non-cyclized forms of the chimeric proteins of the invention may be driven by any of a number of promoters. For example, viral promoters such as the ³⁵S RNA and 19S RNA promoters of CaMV (Brisson et al. 1984, Nature 310:511-514), or the coat protein promoter of TMV (Takamatsu et al. 1987, EMBO J. 6:307-311) may be used; alternatively, plant promoters such as the small subunit of RUBISCO (Coruzzi et al. 1984, EMBO J. 3:1671-1680; Broglie et al. 1984, Science 224:838-843) or heat shock promoters, e.g., soybean hsp17.5-E or hsp17.3-B (Gurley et al. 1986, Mol. Cell. Biol. 6:559-565) may be used. These constructs can be introduced into plant cells using Ti plasmids, R1 plasmids, plant virus vectors, direct DNA transformation, microinjection, electroporation, etc. For reviews of such techniques see, e.g., Weissbach & Weissbach 1988, Methods for Plant Molecular Biology, Academic Press, NY, Section VIII, pp. 421-463; and Grierson & Corey 1988, Plant Molecular Biology, 2^{nd} ed., Blackie, London, Ch. 7-9.

In one insect expression system that may be used to produce the chimeric proteins of the invention, Autographa californica nuclear polyhidrosis virus (ACNPV) is used as a vector to express the foreign genes. The virus grows in Spodoptera frugiperda cells. A coding sequence may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (see, e.g., Smith et al. 1983, J. Virol. 46:584; U.S. Pat. No. 4,215,051). Further examples of this expression system may be found in Ausubel et al., eds. 1989, Current Protocols in Molecular Biology, Vol. 2, Greene Publish. Assoc. & Wiley Interscience.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g. region E1 or E3) will result in a recombinant virus that is viable and capable of expressing peptide in infected hosts (see, e.g., Logan & Shenk 1984, Proc. Natl. Acad. Sci. (USA) 81:3655-3659). Alternatively, the vaccinia 7.5 K promoter may be used (see, e.g., Mackett et al. 1982, Proc. Natl. Acad. Sci. (USA) 79:7415; Mackett et al. 1984, J. Virol. 49:857; Panicali et al. 1982, Proc. Natl. Acad. Sci. (USA) 79:4927).

In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g. region E1 or E3) will result in a recombinant virus that is viable and capable of expressing peptide in infected hosts (see, e.g., Logan & Shenk 1984, Proc. Natl. Acad. Sci. (USA) 81:3655-3659). Alternatively, the vaccinia 7.5 K promoter may be used (see, e.g., Mackett et al. 1982, Proc. Natl. Acad. Sci. (USA) 79:7415-7419; Mackett et al. 1984, J. Virol. 49:857-864; Panicali et al. 1982, Proc. Natl. Acad. Sci. (USA) 79:4927).

Host cells containing DNA constructs of the chimeric protein are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals and growth factors. Optionally the media can contain bovine calf serum or fetal calf serum. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum-containing or serum-free media (e.g. MEM, DMEM). Selection of a medium appropriate for the particular cell line used is within the level of ordinary skill in the art.

The recombinantly produced chimeric protein of the invention can be isolated from the culture media. The culture medium from appropriately grown transformed or transfected host cells is separated from the cell material, and the presence of chimeric proteins is demonstrated. One method of detecting the chimeric proteins, for example, is by the binding of the chimeric proteins or portions of the chimeric proteins to a specific antibody recognizing the chimeric protein of the invention (e.g., an anti-Fc antibody). An anti-chimeric protein antibody may be a monoclonal or polyclonal antibody raised against the chimeric protein in question. For example, the chimeric protein can contain a portion of an immunoglobulin constant region. Antibodies recognizing the constant region of many immunoglobulins are known in the art and are commercially available. An antibody can be used to perform an ELISA or a western blot to detect the presence of the chimeric protein of the invention.

The chimeric protein of the invention can be synthesized in a transgenic animal, such as a rodent. The term “transgenic animals” refers to non-human animals that have incorporated a foreign gene into their genome. Because this gene is present in germline tissues, it is passed from parent to offspring. Exogenous genes are introduced into single-celled embryos (Brinster et al. 1985, Proc. Natl. Acad. Sci. USA 82:4438). Methods of producing transgenic animals are known in the art, including transgenics that produce immunoglobulin molecules (Wagner et al. 1981, Proc. Natl. Acad. Sci. USA 78:6376; McKnight et al. 1983, Cell 34:335; Brinster et al. 1983, Nature 306:332; Ritchie et al. 1984, Nature 312:517).

The chimeric protein of the invention can also be produced by a combination of synthetic chemistry and recombinant techniques. For example, the portion of an immunoglobulin constant region can be expressed recombinantly as described above. The viral fusion inhibitor can be produced using known chemical synthesis techniques (e.g., solid phase synthesis).

The portion of an immunoglobulin constant region can be ligated to the viral fusion inhibitor using appropriate ligation chemistry. For example, the viral fusion inhibitor can be chemically synthesized with an N terminal cysteine. The sequence encoding a portion of an immunoglobulin constant region can be sub-cloned into a vector encoding intein linked to a chitin binding domain. The intein can be linked to the C terminus of the portion of an immunoglobulin constant region which can rearrange to form an IgG/Fc C terminal thioester. Alternatively, the viral fusion inhibitor can be synthesized with an C terminal thioester and the portion of an immunoglobulin constant region can be generated with an N terminal cysteine. The viral fusion inhibitor and portion of an immunoglobulin constant region can be reacted together such that nucleophilic rearrangement occurs and the viral fusion inhibitor is covalently and irreversibly linked to the portion of an immunoglobulin constant region via an amide bond. (Dawson et al. 2000, Annu. Rev. Biochem. 69:923). The chimeric protein synthesized this way can optionally include a linker peptide between the portion of an immunoglobulin constant region and the viral fusion inhibitor. The linker can for example be synthesized on the N terminus of the viral fusion inhibitor. Linkers can include peptides and/or organic molecules (e.g. polyethylene glycol and/or short amino acid sequences). This combined recombinant and chemical synthesis allows for the rapid screening of potential viral fusion inhibitors and linkers to optimize desired properties of the chimeric protein of the invention, e.g., viral fusion inhibitor activity, biological half-life, stability, binding to serum proteins or some other property of the chimeric protein. The method also allows for the incorporation of non-natural amino acids into the chimeric protein of the invention which may be useful for optimizing a desired property of the chimeric protein of the invention. If desired, the chimeric protein produced by this method can be refolded to a biologically active conformation using conditions known in the art, e.g., reducing conditions and then dialyzed slowly into PBS. It will be recognized by the skilled artisan that there are other methods of making protein conjugates (see, e.g., Aslam and Dent 1998, Bioconjugation: Protein Coupling Techniques For The Biomedical Sciences, Macmillan Reference, London).

F. Methods of Using Chimeric Proteins

The chimeric proteins of the invention have many uses as will be recognized by one skilled in the art, including, but not limited to methods of treating a subject and methods of inhibiting viral fusion with a target cell.

1. Methods of Treating a Patient

The invention relates to a method of treating a subject having a viral infection or exposed to a virus comprising administering a therapeutically effective amount of at least one chimeric protein wherein the chimeric protein comprises at least a portion of an immunoglobulin constant region and at least one viral fusion inhibitor. In one embodiment the subject is infected with HIV, such as HIV-1 or HIV-2.

The chimeric protein of the invention prevents or inhibits viral entry into target cells, thereby stopping, preventing, or limiting the spread of a viral infection in a subject and decreasing the viral burden in an infected subject. By linking a portion of an immunoglobulin constant region to a viral fusion inhibitor the invention provides a chimeric protein with viral fusion inhibitory activity with greater stability and greater bioavailability compared to viral fusion inhibitors alone, e.g., T20, T21, T1249. Thus, in one embodiment the viral fusion inhibitor decreases or prevents HIV infection of a target cell, e.g., HIV-1.

The invention provides for a chimeric protein which decreases or prevents viral penetration of a cellular membrane of a target cell. The chimeric protein of the invention can prevent the formation of syncytia between at least two susceptible cells. The chimeric protein of the invention can prevent the joining of a lipid bilayer membrane of a eukaryotic cell and an a lipid bilayer of an enveloped virus.

a. Conditions that May be Treated

The chimeric protein of the invention can be used to inhibit or prevent the infection of any target cell by any virus. In one embodiment, the virus is an enveloped virus such as, but not limited to HIV, SIV, measles, influenza, Epstein-Barr virus, respiratory syncytia virus, or parainfluenza virus. In another embodiment, the virus is a non-enveloped virus such as rhino virus or polio virus.

The chimeric protein of the invention can be used to treat a subject already infected with a virus. The subject can be acutely infected with a virus. Alternatively, the subject can be chronically infected with a virus. The chimeric protein of the invention can also be used to prophylactically treat a subject at risk for contracting a viral infection, e.g., a subject known or believed to in close contact with a virus or subject believed to be infected or carrying a virus. The chimeric protein of the invention can be used to treat a subject believed to be exposed to a virus, but who has not yet been positively diagnosed.

In one embodiment, the invention relates to a method of treating a subject infected with HIV comprising administering to the subject a therapeutically effective amount of a chimeric protein wherein the chimeric protein comprises an Fc fragment of an IgG and the viral fusion inhibitor comprises T20.

b. Treatment Modalities

The chimeric protein of the invention can be administered intravenously, subcutaneously, sublingually, intramuscularly, orally, buccally, nasally, rectally, or via pulmonary route. The chimeric protein can be implanted within or linked to a biopolymer solid support that allows for the slow release of the chimeric protein.

The dose of the chimeric protein of the invention will vary depending on the subject and upon the particular route of administration used. Dosages can range from 0.1 to 100,000 μg/kg body weight. In one embodiment, the dosing range is 1-10 mg/kg. The protein can be administered continuously or at specific timed intervals. In vitro assays may be employed to determine optimal dose ranges and/or schedules for administration. Many in vitro assays that measure viral infectivity are known in the art. For example, a reverse transcriptase assay, or an rt PCR assay or branched DNA assay can be used to measure HIV concentrations. Additionally, effective doses may be extrapolated from dose-response curves obtained from animal models.

The invention also relates to a pharmaceutical composition comprising a viral fusion inhibitor, at least a portion of an immunoglobulin constant region and a pharmaceutically acceptable carrier or excipient. Examples of suitable pharmaceutical carriers are described in Remington's Pharmaceutical Sciences by E. W. Martin. Examples of excipients can include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g. sorbitol syrup, cellulose derivatives or hydrogenated edible fats), emulsifying agents (lecithin or acacia), non-aqueous vehicles (e.g. almond oil, oily esters, ethyl alcohol, or fractionated vegetable oils), and preservatives (e.g. methyl or propyl-p-hydroxybenzoates or sorbic acid). The preparations can also include flavoring, coloring and sweetening agents. Alternatively, the composition can be presented as a dry product for constitution with water or another suitable vehicle.

For buccal and sublingual administration the composition may take the form of tablets or lozenges according to conventional protocols.

For administration by inhalation, the compounds for use according to the present invention are conveniently delivered in the form of an aerosol spray from a pressurized pack or nebulizer, with a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can be formulated for parenteral administration (i.e., intravenous or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

The pharmaceutical composition can also be formulated for rectal administration as a suppository or retention enema, e.g., containing conventional suppository bases such as cocoa butter or other glycerides.

c. Combination Therapy

In another embodiment the invention relates to a method of treating a subject infected with HIV comprising administering a therapeutically effective amount of at least one chimeric protein comprising an HIV fusion inhibitor and at least a portion of an immunoglobulin constant region in combination with at least one other anti-HIV agent. Said other anti-HIV agent can be any therapeutic with demonstrated anti-HIV activity. Said other anti-HIV agent can include, as an example, but not as a limitation, a protease inhibitor (e.g. Amprenavir®, Crixivan®, Ritonivir®, a reverse transcriptase nucleoside analog (e.g. AZT, DDI, D4T, 3TC, Ziagen®), a normucleoside analog reverse transcriptase inhibitor (e.g. Sustiva®), another HIV fusion inhibitor, a neutralizing antibody specific to HIV, an antibody specific to CD4, a CD4 mimic, e.g., CD4-IgG2 fusion protein (U.S. patent application Ser. No. 09/912,824) or an antibody specific to CCR5, or CXCR4, or a specific binding partner of CCR5, or CXCR4.

2. Methods of Inhibiting Viral Fusion with a Target Cell

The invention also relates to an in vitro method of inhibiting HIV fusion with a mammalian cell comprising combining the mammalian cell with at least one chimeric protein, wherein the chimeric protein comprises at least a portion of an immunoglobulin constant region and an HIV inhibitor. The mammalian cell can include any cell or cell line susceptible to infection by HIV including but not limited to primary human CD4⁺ T cells or macrophages, MOLT-4 cells, CEM cells, AA5 cells or HeLa cells which express CD4 on the cell surface.

G. Kits and Methods of Detection

The invention provides a kit for the detection of HIV in a sample. The kit can include a container and a chimeric protein comprising a viral fusion inhibitor, and at least a portion of an immunoglobulin. In one embodiment, the portion of an immunoglobulin constant region is an Fc fragment. In another embodiment, the portion of an immunoglobulin constant region is an FcRn binding partner. The chimeric protein can be provided in an appropriate buffer or solvent. The buffer can be an aqueous buffer, e.g., PBS or alternatively the chimeric protein can be lyophilized. The kit can also provide instructions for measuring for the presence of HIV in a sample, e.g., a) contacting a first aliquot of a sample with the chimeric protein of the invention and measuring for the presence of HIV, and b) comparing the amount of HIV in a) with the amount of HIV in a second aliquot of the sample not contacted with the chimeric protein of the invention. The kit can optionally provide an aliquot of cells susceptible to HIV infection.

The invention also provides for a kit for testing the drug sensitivity of an HIV specimen comprising a chimeric protein comprising an HIV fusion inhibitor and at least a portion of an immunoglobulin constant region in a container. In one embodiment, the portion of an immunoglobulin constant region is an Fc fragment. In another embodiment, the portion of an immunoglobulin constant region is an FcRn binding partner. In one embodiment, the HIV fusion inhibitor is an HIV-1 fusion inhibitor. The HIV fusion inhibitor can be, e.g., T20, T21, T1249. The chimeric protein can be provided in an appropriate buffer or solvent. The buffer can be an aqueous buffer, e.g., PBS or alternatively the chimeric protein can be lyophilized. The kit can optionally provide a strain of HIV resistant to a fusion inhibitor. The kit can optionally provide a strain of virus sensitive to a fusion inhibitor.

### EXAMPLES

### Example 1

### Generation of Recombinant Fc-T20/T20-Fc

Recombinant chimeric proteins comprised of T20 and human Fc were made using nested PCR. Pfu DNA polymerase (Stratagene, La Jalla Calif.) was used in all amplifications. Composition of the PCR reactions were prepared based on the manufacturer's manual. Rounds one and two of the nested PCR were performed with 10 cycles, (94° C. for 45 seconds, 45° C. for 45 seconds, and 72° C. for 2 minutes) while round three was performed in 30 cycles using the same cycling parameters as rounds 1 and 2. All sequences were confirmed by DNA sequencing.

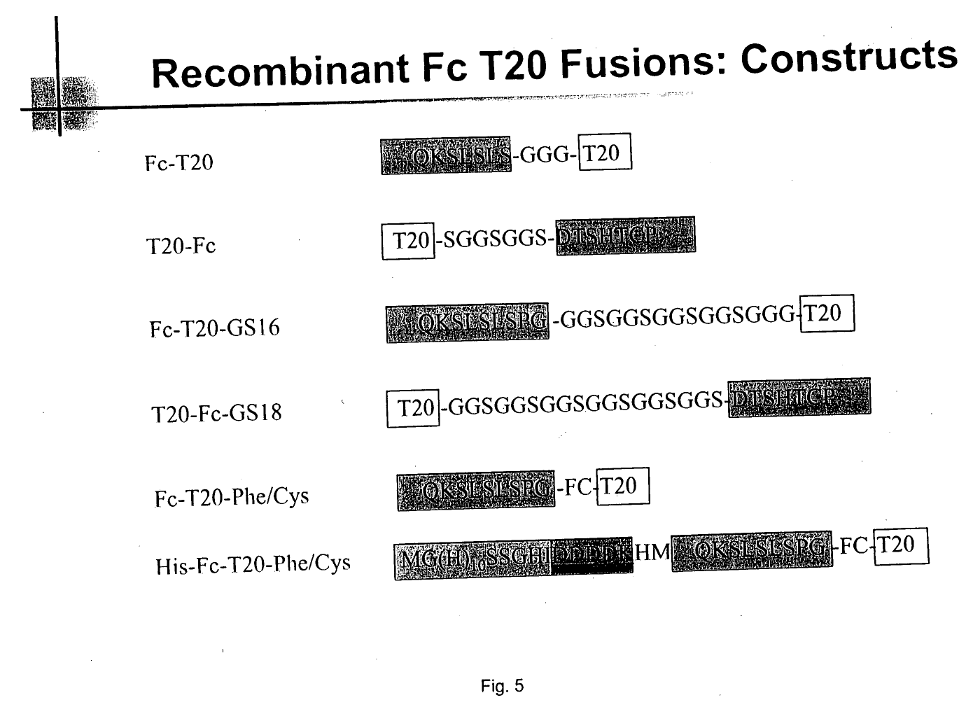

A. Construction of Fc-T20 (FIG. 5)

The first round of nested PCR used human Fc as the template and primers 3′ first Fc-T20 (5′-GATCAGGCTGTGGATCAGGGAAGTGTAGCC ACCGCCACCCGGAGACAGGGAGAGGCTTTTC-3′) (SEQ ID NO: 30) and 5′ Fc-T20 (5′-TCGCCTGCTCTTCC AACGCCGACAAAACTCACACA-3′) (SEQ ID NO: 31). The resulting PCR product was used as a template in the second round of PCR with primers 3′ second Fc-T20 (5′-GCAGTT CCTGTTCGTTCTTTTCCTGCTGGTTCTGAGATTCTTCGATCAGGCTGTGG ATCAGG-3′) (SEQ ID NO: 32) and 5′ Fc-T20. The product from this PCR was then used as a template in the third and last round using primers 3′ third Fc-T20 (5′-ACCACCCTGCAGTCAG AACCAGTTCCACAGAGAG GCCCATTTGTCCAGCTCGAGCAGTTCCTGTT CGTTCT-3′) (SEQ ID NO: 33) and 5′Fc-T20.

B. Construction of T20-Fc (FIG. 5)

The first round of nested PCR used human Fc as the template and primers 3′ first T20-Fc (5′-CCGGAGACAGGGAGAGGCTTTTCTGCGTG-3′) (SEQ ID NO: 34) and 5′ first T20-Fc (5′-CTGGACAAATGGGCCTCTCTGTGGAACTGGTTCGG CGGTGGTGGCGACAAAACTCACACA-3′) (SEQ ID NO: 35). The resulting PCR product was used as the template in the next round with primers 5′ second T20-Fc (5′-GAAGAATC TCAGAACCAGCAGGAAAAGAACGAACAGGAACTGCTCGAGCTGGACAAA TGGGCC-3′) (SEQ ID NO: 36) and 3′ second T20-Fc (5′-CTACTACTGCAGTTAACCCGGAGACAGG-3′) (SEQ ID NO: 37). The PCR product from this round was then used as the template for the last round with primers 5′ third T20-Fc (5′-GGTGGTTGCTCTTCCAACTACACTTCCC TGATCCACAGCCTGATCGAAGAATCTCAGAACCAGC-3′) (SEQ ID NO: 38) and 3′ second T20-Fc.

The primers also added a glycine linker between T20 and Fc as well as a Pst1 and Sap 1 restriction sites to enable cloning of Fc-T20/T20-Fc in either pTwin1 or pTYB11 vectors (New England Biolabs, Beverly, Mass.). Use of these vectors facilitated expression of Fc-T20/T20-Fc with a cleavable intein tag in E. coli and a chitin binding domain for easy purification using chitin beads as instructed in the New England Biolabs manual. On-column cleavage was done by incubation of the fusion protein-chitin beads with 50 mM dithiothreitol at room temperature for 24 hours. Under these conditions, only 30% Fc-T20 was cleaved from the intein tag.

### Example 2

### Generation of Recombinant Fc-T20/T20-Fc for Expression in CHO Cells

A. Fc-T20 for Expression in DG44 (CHO)

PCR was performed to amplify the gene for T20 using template intein-Fc-T20 in pTYB11 described in Example 1. The following cycling conditions were used. One cycle of 94° C. for 45 seconds, followed by 30 cycles of 94° C. for 45 seconds, 55° C. for 45 seconds, 72° C. for 2 minutes, and finally, one cycle of 72° C. for 10 minutes. The primers also contained restriction sites for Blp1 and EcoR1. The primers used were: (5′-TTTTGAATTCTCAGAACC AGT TCCACAGAGAGGC-3′ (SEQ ID NO: 39) and 5′-TGTCGCTGAGCGGCGGTGGCTACACTTCCC TG-3′) (SEQ ID NO: 40). The T20 PCR product was ligated into Blp1/EcoR1 digested vector (pEdDC with an Fc gene inserted) producing Fc-T20-pEdDC.

B. T20-Fc for Expression in DG44 (CHO)

For the T20-Fc orientation, the primers for PCR contained restriction sites for Not1 and BspE1. Two rounds of nested PCR were performed using the following conditions: one cycle of 94° C. for 45 seconds, followed by 30 cycles of 94° C. for 45 seconds, 55° C. for 45 seconds, 72° C. for 2 minutes, and finally, one cycle of 72° C. for 10 minutes. The first round utilized intein-Fc-T20 in pTYB11 as the template and primers 5′ first X-Fc (5′-GACTCAGACCCGCGCCTACACTTCCCTGATCCACAG-3′) (SEQ ID NO: 41) and 3′BspE1 (5′-TTTTTCCGGAGAACCAGTTCCACAGAGA-3′) (SEQ ID NO: 42). The resulting PCR product was used as the template for the next round with primers 5′ second X-Fc (5′-TATTGGCGGCCGCCCTGGCTCCGACTCAGACCCGCG-3′) (SEQ ID NO: 43) and 3′BspE1. The digested PCR product was inserted into NotI/BspEI-digested vector EMP1-Fc-pEdDC producing T20-Fc-pEdDC

C. T20-Fc-G518 and Fc-T20GS16 for Expression in DG44 (CHO)

A similar strategy was employed to construct Fc-T20 with a longer linker between Fc and T20 (Fc-T20-GS16), (FIG. 5), and T20-Fc with a longer linker (T20-Fc-GS18). In both cases the linker was added through two rounds of nested PCR For Fc-T20-GS16, the template was Fc-T20-pEdDC for the first round of nested PCR and the primers were 3′Fc-T20-GGS (5′-CGCACACCG GCCTTCGCA ATTCCAA-3′) (SEQ ID NO: 44) and 5′-1st-Fc-T20-GGS (5′-CGGCTCCGGC GGCTCTGGTGGCTCT GGCGGTGGCTA-3′) (SEQ ID NO: 45). The next round utilized the PCR product from the first round and primers 3′Fc-T20-GGS and 5′-2nd-Fc-T20-GGS (5′-CTCTCGCTGAGCCCCGGTGGCGGTTCCGGCGGCTCC-3′) (SEQ ID NO: 46). The final PCR product was digested with Blp1 and EcoR1 and ligated into similarly digested Fc-T20-pEdDC (with the T20 fragment removed), to form vector T20-GS16-pEdDC.

For T20-Fc-GS18, the template was T20-Fc-pEdDC in the first PCR and the primers were 5′T20-Fc-GGS (5′CTGCTGTTGGCGGCCGCCCTGGCTC-3′) (SEQ ID NO: 47) and 3′-1st-T20-Fc-GGS (5′CCGCTACCACCGCTGCCACCGAACCAGTTCCACAGA-3′) (SEQ ID NO: 48). The primers for the last nested PCR were 5′T20-Fc-GGS and 3′-2nd T20-Fc-GGS (5′-TCACATCCGGAACCGCCGCTACCGCCGCTACCACCG-3′) (SEQ ID NO: 49). The final PCR product was digested with BspE1 and Not1 and inserted into similarly digested T20-Fc-pEdDC to form vector T20-GS18-pEdDC.

D. Fc-T20-Phe-Cys for Expression in DG44 (CHO Cells)

Fc-T20-Phe-Cys is a recombinant version of the protein produced through intein-mediated chemical ligation. The template for PCR was Fc-T20-pEdDC and primers used were 3′Fc-T20-GGS and 5′Fc-T20-IPL (5′-AGAG CTCGCTGAGCCCGGGCTTTTGCTACACTTCCCT-3′) (SEQ ID NO: 50). The PCR product was digested with Blp1 and EcoR1 and cloned into similarly digested Fc-T20-pEdDC (with the T20 gene removed) to form vector Fc-T20-Phe-Cys-pEdDC.

### Example 3

### Cloning of Fc-T20 Constructs in E. coliVector

Both Fc-T20-GS16 and Fc-T20-PheCys were also cloned into an E. coli vector. The Blp1 and Not 1-digested PCR products for both constructs were inserted into a derivative of pThioHisA vector (Invitrogen, Carlsbad, Calif.). This derivative vector contains an Fc gene and restriction sites Blp1 and Not 1 to facilitate cloning of several proteins fused to the Fc fragment.

### Example 4

### Expression of T20 Constructs in CHO Cells

The day before transfection, 2.5×10⁶ CHO DG44 cells were seeded into a 100 mm tissue culture dish. Cells were incubated at 37° C. and 5% CO₂ overnight. 10 μg of DNA was diluted with cell growth medium (Minimum Essential Medium with ribonucleosides and deoxyribonucleosides) with no fetal bovine serum (FBS) and no antibiotics to a total volume of 300 μL. 60 μL of Superfect Transfection reagent (Qiagen Valencia, Calif.) was added. The tube was incubated for 5-10 minutes at room temperature to allow complex formation. Then 3 mL of cell growth medium (with 5% FBS) was added to the tube. Meanwhile, the medium from the dish was aspirated and the cells washed with PBS. The contents of the tube was transferred to the dish. The cells were incubated with the Superfect mixture for 2-3 hours at 37° C. and 5% CO₂. The Superfect mixture was then aspirated and the cells washed three times with PBS. Fresh cell growth medium with FBS was added. Cells were incubated for 48 hours. Transient expression of the T20 chimeric protein was checked by Western blot detection of the Fc fusion protein. The antibody used for detection was goat anti-human Fc-HRP (1:25,000 dilution,) (Pierce, Rockford, Ill.). Cells were also passaged into selective medium (Minimum Essential Medium without ribonucleosides and deoxyribonucleosides with 5% dialyzed FBS). Once stable transformants were established, protein expression was enhanced by methotrexate amplification.

### Example 5

### Purification of T20 Chimeric Protein from DG44

The Fc-T20 chimeric proteins produced in CHO DG44 cells were secreted into the culture medium as a result of the signal peptide which was engineered into the construct and cleaved by the cell to form the mature protein. Stable cell lines were grown in roller bottles. Culture supernatant was collected daily for 10 days after seeding. Supernatant was filtered and loaded onto a Protein A Sepharose column (Pharmacia Peapack, N.J.). The column was washed with PBS. The Fc chimeric protein was eluted from the column under acidic condition (100 mM Glycine, pH 2.7). The eluents were neutralized by 1 M Tris, pH 8. Fractions containing the chimeric protein were dialyzed in several changes of PBS.

In cases where Fc-T20 aggregates occurred along with the correct dimeric form, an extra purification step was added using a Superdex 200 size exclusion chromatography column (Amersham, Uppsala, Sweden).

### Example 6

### Peptide Synthesis

The N-terminal cysteine-containing peptides were synthesized on an Advanced Chemtech 396 Ω synthesizer (Advanced Chemtech, Louisville, Ky.) using the standard Fmoc/tBu protocols (W. C. Chan and P. D. White eds. 2000, Fmoc Solid Phase Peptide Synthesis: A Practical Approach; Oxford University Press Inc., New York), using Rink amide resin (Novabiochem, San Diego Calif.), O-benzotriazol-1-yl-N,N,N′,N′-tetramethyluronium hexafluorophosphate (Novabiochem, San Diego, Calif.) as the coupling reagent, diisopropylehtylamine (DIEA), (Sigma-Aldrich, St. Louis, Mo.) as the base, and N,N-dimethylformamide as the solvent (EM Science, Kansas City, Mo.). In some cases, N-Fmoc-amido-dPEG4-acid (Quanta Biodesign, Powell, Ohio) was used as a spacer between the N-terminal cysteine and the rest of the peptide. Peptides were cleaved from the resin using 95% trifluoroacetic acid; 2.5% ethanedithiol; 1.5% triisopropylsilane and 1% water for 3 hours. Peptides were precipitated with ice-cold ether, centrifuged and triturated three times with ether. Peptides were purified by reversed phase HPLC on a Waters Prep600 System (Millford, Mass.) using a C18 column (Phenomenex, Torrence Calif., 250 mm×21.2 mm) and gradients of acetonitrile in water with 0.1% TFA as the eluent. The appropriate fractions were pooled and lyophilized to give white powders. Peptide identity and purity was confirmed with reversed phase analytical HPLC (Phenomenex, Torrence, Calif.) using a 250 mm×2 mm column coupled with electrospray mass spectrometry (Mariner ES-MS) (Applied Biosystems, Foster City, Calif.).

The C-terminal thioester peptides were synthesized as described above except for the following: a modified resin was used to allow for selective cleavage of the protected peptide off the resin. Mezo et al. 2001, J. Am. Chem. Soc., 123:3885. Fmoc-Gly-TGT resin (Novabiochem, San Diego, Calif.) was used for the synthesis. In some cases, N-Fmoc-amido-dPEG4-acid (Quanta Biodesign, Powell, Ohio) was used as a spacer between the C-terminal Gly residue and the rest of the peptide. Selective cleavage of the protected peptide from the resin was achieved using 50% acetic acid, 40% dichloromethane and 10% methanol (10 mL). The solvent was then added to 100 mL hexanes, then evaporated in vacuo. An additional 100 mL hexanes was added and evaporated in vacuo. The crude protected peptide was then dissolved in tetrahydrofuran, and to this solution was added benzyl mercaptan (4 equivalents), diisopropylethylamine (8 equivalents) and O-benzotriazol-1-yl-N,N,N′,N′-tetramethyluroniumhexafluorophosphate (4 equivalents). The reaction was stirred for 16 hours, at which point the solvent was evaporated and the peptide was subjected to the full peptide cleavage and purification using the same conditions described above.

### Example 7

### Protein Expression and Preparation of Fc-MESNA

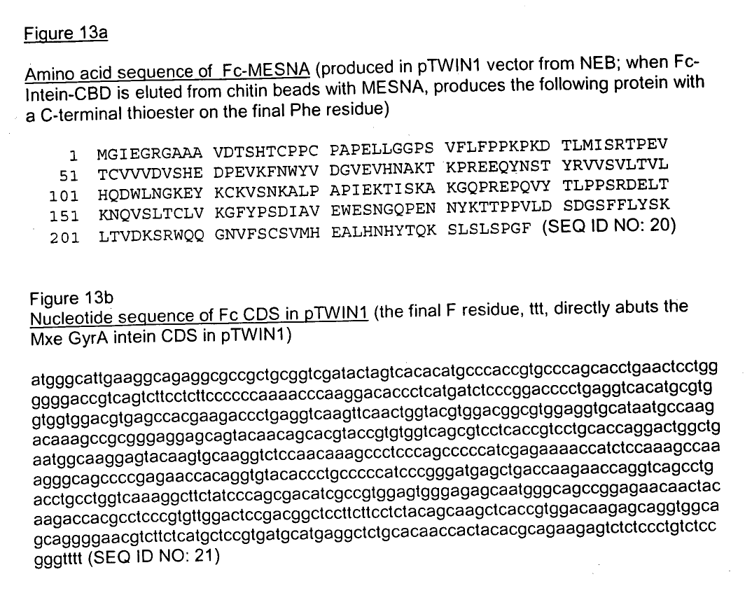

The coding sequence for Fc (the constant region of human IgG1) was obtained by PCR amplification from an Fc-containing plasmid using standard conditions and reagents, following the manufacturer's recommended procedure to subclone the Fc coding sequence NdeI/SapI. Briefly, the primers 5′-GTGGTCATA TGGGCATTGAAGGCAGAGGCGCCGCTGCGGTCG-3′ (SEQ ID NO: 51) and 5′-GGTGGTTGC TCTTCCGCAAAAACCCGGAGACAGGGAGAGACTCTTCTGCG-3′ (SEQ ID NO: 52) were used to amplify the Fc sequence from 500 ng of the plasmid pED.dC.Epo-Fc using Expand High Fidelity System (Boehringer Mannheim, Basel Switzerland) in a RapidCycler thermocycler (Idaho Technology Salt Lake City, Utah), denaturing at 95° C. for 2 minutes followed by 18 cycles of 95° C. for 0 sec, 55° C. for 0 sec, and 72° C. for 1 minute with a slope of 4, followed by 72° C. extension for 10 minutes. The PCR product was subcloned into an intermediate cloning vector and sequenced fully, and then subcloned using the NdeI and SapI sites in the pTWIN1 vector following standard procedures Sambrook J., Fritsch, E. F. and Maniatis, T. 1989, Molecular Cloning: A Laboratory Manual, 2^{nd} ed.; Cold Spring Harbor, N.Y.: Cold Spring Harbor Laboratory Press. This plasmid was then transformed into BL21 (DE3) pLysS cells using standard methods. Id. A 1 liter culture of cells was grown to an absorbance reading of 0.8 AU at 37° C., induced with 1 mM isopropyl beta-D-1-thiogalactopyranoside, and grown overnight at 25° C. Cells were pelleted by centrifugation, lysed in 20 mM Tris 8.8/1% NP40/0.1 mM phenylmethanesulfonyl fluoride/1 μg/ml Benzonase (Novagen Madison, Wis.), and bound to chitin beads (New England Biolabs; Beverly, Mass.) overnight at 4° C. Beads were then washed with several column volumes of 20 mM Tris 8.5/500 mM NaCl/1 mM EDTA, and then stored at −80° C. Purified Fc-MESNA was generated by eluting the protein from the beads in 20 mM Tris 8.5/500 mM NaCl/1 mM EDTA/500 mM mercapto ethanol sulfonic acid (MESNA), and the eluate was used directly in the coupling reaction, below (FIG. 13).

### Example 8

### Coupling of Fc-MESNA to Cys-T20 Peptides

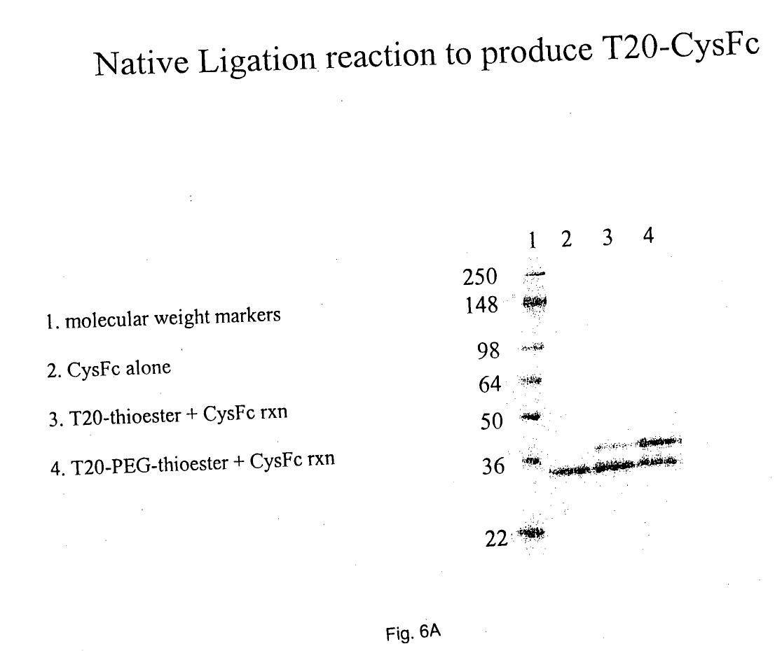

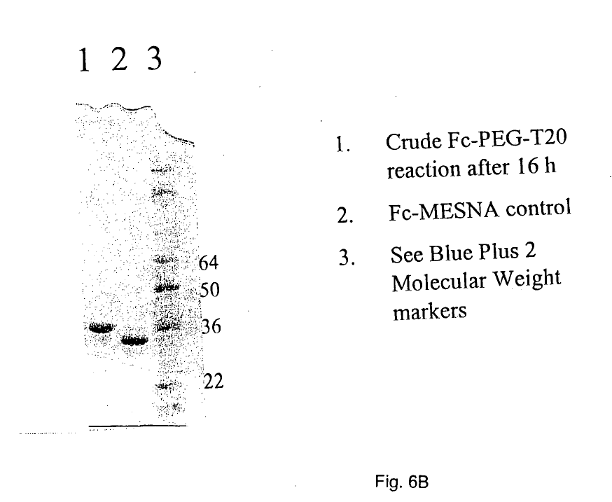

Fc-MESNA (2.6 mg/mL in 50 mM Tris pH 8, 500 mM MESNA; 20.5 mL used) and Cys-PEG-T20 (28.4 mg, 4799 g/mol, 3 equivalents) were incubated for 16 hours at room temperature. Analysis by SDS-PAGE (Tris-Gly gel) using reducing sample buffer indicated the presence of a new band approximately 5 kDa larger than the Fc control (>90% conversion to the conjugate). (FIG. 6B).

### Example 9

### Refolding of Fc-PEG-T20 (Fc-299)

To Fc-PEG-T20 in 50 mM Tris pH 8 buffer was added urea for a final concentration of 8 M urea. The protein was then dialyzed into 2 liters of denaturing buffer consisting of 8 M urea, 50 mM Tris pH 8, 1 mM EDTA, 5 mM reduced glutathione (GSH) for 2 hours. The sample was then changed into fresh denaturing buffer (2 liters) and dialyzed for 16 hours. The sample was changed one more time into fresh denaturing buffer (2 liters) and oxidized gluathione (GSSG) was added to the dialysis denaturing buffer for a final concentration of 2.5 mM GSSG. The protein was then slowly refolded following modified literature procedures. Maeda et al. 1996, Protein Eng. 9:95; Ueda et al. 1997, Cell Mol. Life Sci. 53:929. With the protein still suspended in dialysis tubing in 1 liter of redox denaturing dialysis buffer, a new buffer of 50 mM Tris pH 8, 1 mM EDTA, 5 mM GSH and 2.5 mM GSSG was prepared (4 liters) and pumped into the 1 liter denaturing buffer at a rate of 1 mL/min for 16 hours using a peristaltic pump. Excess buffer was displaced such that the volume of denaturing buffer remained 1 liter. After 16 hours, the rate of addition was increased to 3 mL/min until all of the 4 liters of renaturation buffer was consumed. The protein was then placed into 2 liters of 50 mM Tris pH 8, 5 mM GSH, 2.5 mM GSSG, 1 mM EDTA for 2 hours and replaced with fresh identical buffer and dialyzed for 2 hours. The protein was then dialyzed into 4 liters of PBS for 2 hours, and then into 4 liters of fresh PBS for 16 hours at 4° C.

The protein was purified by size exclusion chromatography using a Superdex 200 column in PBS. The appropriate fractions were collected and concentrated using spin concentrators to attain a protein concentration of 2.3 mg/mL. Total yield was 13.9 mg (26% based on Fc-MESNA protein). Analysis by SDS-PAGE showed a single dominant band on a reducing gel at approximately 5 kDa larger than the Fc control.

### Example 10

### Protein Expression of CysFc

Using PCR and standard molecular biology techniques, a mammalian expression construct was generated such that the coding sequence for the human IFNα signal peptide was directly abutted against the coding sequence of Fc beginning at the first cysteine residue (Cys 226, EU Numbering). Sambrook J., Fritsch, E. F. and Maniatis, T. 1989, Molecular Cloning: A Laboratory Manual, 2^{nd} ed.; Cold Spring Harbor, N.Y.: Cold Spring Harbor Laboratory Press. Upon signal peptidase cleavage and secretion from mammalian cells, an Fc protein with an N-terminal cysteine residue would thus be generated. Briefly, the primers IFNa+Sig-F (IFNa+Sig-F: 5′-GCTACTGCAGCCACCATGGCCTTGACCTTTGCTTTAC-3′) (SEQ ID NO: 53) and Cys-Fc-R (5′-CAGTTCCGGAGCTGGGCACGGCGGAGAGCCCACAGAGCAGCTTG-3′) (SEQ ID NO: 54) were used in a PCR reaction to create a fragment linking the IFNα signal sequence with the N terminus of Fc, beginning with Cys 226. 500 ng of pED.dC.native hIFNα Δlinker was added to 25 pmol of each primer in a PCR reaction with Expand High Fidelity System (Boehringer Mannheim, Indianapolis, Ind.) according to manufacturer's standard protocol. The reaction was carried out in a MJ Thermocycler using the following cycles: 94° C. 2 minutes; 30 cycles of (94° C. 30 seconds, 50° C. 30 seconds, 72° C. 45 seconds), and finally 72° C. 10 minutes. The expected sized band (˜112 bp) was gel purified with a Gel Extraction kit (Qiagen, Valencia Calif.), digested with the PstI and BspEI restriction enzymes, gel purified, and subcloned into the corresponding sites pED.dC.native hIFNα Δlinker to generate pED.dC.Cys-Fc.

This pED.dC.CysFc expression plasmid, which contains the mouse dihydrofolate reductase (dhfr) gene, was transfected into CHO DG44 (dhfr deficient) cells using Superfect reagent (Qiagen; Valencia, Calif.) according to manufacturer's protocol, followed by selection for stable transfectants in αMEM (without nucleosides) tissue culture media supplemented with 5% dialyzed FBS and penicillin/streptomycin antibiotics (Invitrogen; Carlsbad, Calif.) for 10 days. The resulting pool of stably transfected cells were then amplified with 50 nM methotrexate to increase expression. Approximately 2×10⁷ cells were used to inoculate 300 ml of growth medium in a 1700 cm² roller bottle (Corning, Corning, N.Y.). The roller bottles were incubated in a 5% CO₂ at 37° C. for approximately 72 hours. Then the growth medium was exchanged with 300 ml serum-free production medium (DMEM/F12 with 5 μg/ml bovine insulin and 10 μg/ml Gentamicin). The production medium (conditioned medium) was collected everyday for 10 days and stored at 4° C. Fresh production medium was added to the roller bottles after each collection and the bottles were returned to the incubator. Prior to chromatography, the medium was clarified using a Supor Cap-100 (0.8/0.2 μm) filter from Pall Gelman Sciences (Ann Arbor, Mich.). All of the following steps were performed at 4° C. The clarified medium was applied to Protein A Sepharose, washed with 5 column volumes of 1×PBS (10 mM phosphate, pH 7.4, 2.7 mM KCl, and 137 mM NaCl), eluted with 0.1 M glycine, pH 2.7, and then neutralized with 1/10 volume of 1 M Tris-HCl, pH 9.0. Protein was dialyzed into PBS and used directly in conjugation reactions.

### Example 11

### Coupling of T20-Thioesters to CysFc

CysFc (4 mg, 3.2 mg/ml final concentration) and either T20-thioester or T20-PEG-thioester (2 mg, approximately 5 molar equivalents) were incubated for 16 hours at room temperature in 0.1 M Tris 8/10 mM MESNA. Analysis by SDS-PAGE (Tris-Gly gel) using reducing sample buffer indicated the presence of a new band approximately 5 kDa larger than the Fc control (>40-50% conversion to the conjugate) (FIG. 6A). Previous N-terminal sequencing of Cys-Fc and unreacted Cys-Fc indicated that the signal peptide is incorrectly processed in a fraction of the molecules, leaving a mixture of (Cys)-Fc, which will react through native ligation with peptide-thioesters, and (Val)-(Gly)-(Cys)-Fc, which will not. As the reaction conditions are insufficient to disrupt the dimerization of the CysFc molecules, this reaction generated a mixture of T20-CysFc:T20-CysFc homodimers, T20-CysFc: Fc monomer/dimer hybrids, and CysFc:CysFc homodimers. This protein was purified using size exclusion chromatography as indicated above, to separate the three species. The result was confirmed by SDS-PAGE analysis under non-reducing conditions.

### Example 12

### Biotinylation of Fc-T20

Fc-T20 was dialyzed exhaustively into PBS. The protein concentration was determined by measuring optical density at 280 nm. A 10 fold molar excess of Sulfo-NHS-Biotin (Pierce, Rockford, Ill.) was added to Fc-T20. The mixture was incubated on ice for 2 hours and dialyzed into PBS. The biotin Fc-T20 conjugate was isolated on a size exclusion column.

### Example 13

### Fc-T20 Binding to FcRn

Neutravidin coated 96 well plates with BSA blocker (Pierce, Rockford, Ill.) were washed twice with Buffer A (PBS, pH 7.4, 0.5% BSA, 0.05%10 Tween 20). Each well was coated with 100 μL of 1 μg/mL biotin-soluble human FcRn (biotin shFcRn) in buffer A. The plates were incubated at 37° C. for 1-2 hours. A solution of 6 nM IgG (Calbiochem, San Diego, Calif.) was prepared in buffer B. A 2× stock of Fc-T20 at a concentration of 2000 nM was prepared in buffer B. The stock was serially diluted to cover a range of 1 mM to 1000 nM by mixing the stock 1:1 with 6 nM IgG solution in the 96 well plate. The plates were rocked at room temperature and then incubated at 37° C. for two hours. The plates were aspirated and 100 μL/well of peroxidase conjugated F(ab¹)₂ goat anti-human Fc (Jackson Immuno Research West Grove, Pa.) diluted 1:10,000 in buffer B was added to each well. The plates were incubated 30 minutes at room temperature and then washed four times with 200 μL/well with ice cold buffer B. 100 μL tetramethylbenzidine peroxidase substrate (TMB) (Pierce, Rockford, Ill.) substrate solution was added to each well and incubated until color develops (5-10 minutes). 100 μL of TMB stop solution (KPL Gaithersburg, Md.) was added to each well and absorbance was measured in a spectrophotometer at 450 nM.

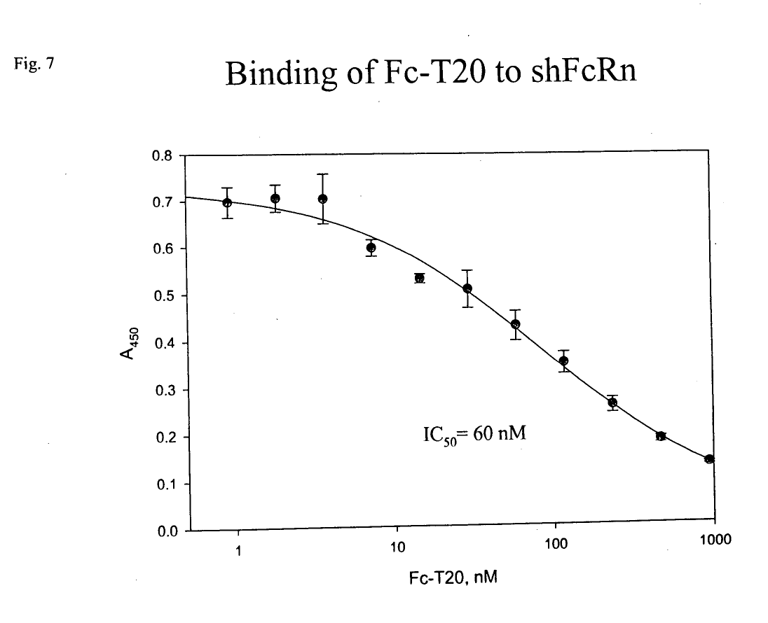

The results indicated Fc-T20 bound to shFcRn with an Ic₅₀ of 20 nM. (FIG. 7)

### Example 14

### Pharmacokinetics of Fc-T20

A. Intravenous Administration of Fc-T20 to Nonhuman Primates

Fc-T20 was administered intravenously to monkeys. For intravenous dosing, biotin-Fc-T20 was administered by a single intravenous bolus injection. The animals were sedated with ketamine (≈10 mg/kg) prior to dosing. The hair from the dosing area were clipped, cleansed with alcohol, and air-dried prior to delivering the dose. This was followed by a 3 mL saline flush. Samples were obtained from the femoral vein at indicated times. The blood was allowed to clot and the serum was separated and frozen until analysis.

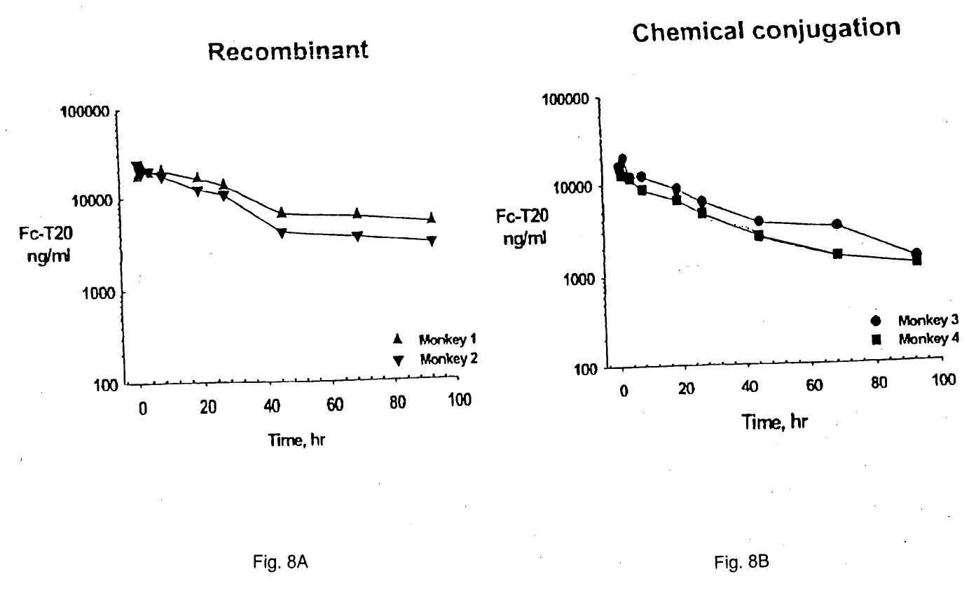

The results indicated recombinantly synthesized Fc-T20 had a serum half life greater than 40 hours. (FIG. 8A). The results indicated chemically conjugated Fc-T20 had a serum half life in monkeys greater than 30 hours. (FIG. 8B).

B. Oral Administration of Fc-T20 to Neonatal Rats

Fc-T20 was formulated in 0.1 M sodium phosphate in 0.9% saline pH 6.5 with 5 mg/ml soybean trypsin inhibitor. The Fc-T20 was administered as a 30-125 μg/ml solution corresponding to a dose of 0.25-1 mg/kg. Ten day old neonatal Sprague Dawley rats received 0.2 ml of the solution containing Fc-T20 by gavage. After dosing, the animals were returned to their mothers. At predetermined time points, 4 animals were euthanized by CO₂ inhalation and blood was collected by cardiac puncture. The blood was allowed to clot and the serum was separated and frozen until analysis.

Fc-T20 serum concentration was determined by ELISA. 96 well plates were coated with 1 μg/ml (50 μl/well) goat anti-human IgG, Fc fragment antibody (Pierce, Rockford, Ill.) in 0.1 M carbonate/bicarbonate buffer pH 9.2 at 37° C. for 1 hour. Plates were blocked with 200 μl/well 2% BSA/PBS at 37° C. for 1 hour. 100 μl/well of sample and standards was added in triplicate and incubated at 37° C. for 1 hour. Samples were initially diluted 1:10 in 2% BSA/PBS. A standard curve of T20-Fc protein was generated by diluting standards to 200 ng/ml, and then down to 1.56 ng/ml in serial twofold dilutions, all in 10% control serum/2% BSA/PBS. Plates were washed 3 times in Tecan plate washer with PBST. 100 μl/well of goat anti-human Fc-HRP conjugate (Pierce, Rockford, Ill.) diluted 1:25,000 in 2% BSA/PBS was added and incubated at room temperature for 1 hour. The plates were washed 3 times in Tecan plate washer with PBST and developed with 100 μl/well of tetramethylbenzidine peroxidase substrate (TMB) (Pierce, Rockford, Ill.) (mixture of equal volumes of solution 1 and 2), incubated 5 minutes at ambient temperature in the dark or until color developed. The reaction was stopped with 100 μl/well of 2 M sulfuric acid. Absorbance was read at 450 nm on plate reader.

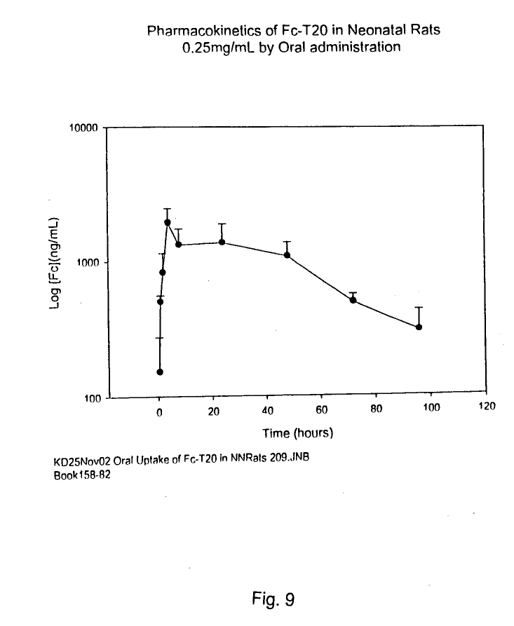

The results indicated orally administered Fc-T20 had a serum half life in neonatal rats of 23 hours. (FIG. 9).

C. Pulmonary Administration of Fc-T20 to Nonhuman Primates

A cuffed endotracheal tube was inserted into the trachea of anesthetized adult Cynomolgus monkeys. The endotracheal tube was connected to a respirator and a nebulizer was connected in-line. A Bird Mark 7A respirator (Bird Products, Palm Springs, Calif.) and an Aeroneb Pro nebulizer (Aerogen, Mountainview, Calif.) was used.

The respirator was set to allow the animal to breath between 25-30 breaths per minute. A solution containing 0.01 to 3 mg/kg of Fc-T20 was loaded in the nebulizer. Two ml of nebulized protein was delivered in 5 minutes. The Fc-T20 was formulated with PBS. Peripheral blood samples were obtained at predetermined time points. The blood was allowed to clot and the serum was separated and frozen until analysis.

Fc-T20 concentrations were measured by steptavidin capture ELISA using biotinylated Fc-T20 and an anti human Fc antibody conjugated to horse radish peroxidase as follows. Neutravidin coated 96 well plates with BSA blocker (Pierce, Rockford, Ill.) were washed twice with Buffer A (PBS, pH 7.4, 0.5% BSA, 0.05%10 Tween 20). Each well was coated with 100 μL of 1 μg/mL biotin-soluble human FcRn (biotin shFcRn) in buffer A. The plates were incubated at 37° C. for 1-2 hours. A solution of 6 nM IgG (Calbiochem, San Diego, Calif.) was prepared in buffer B. A 2× stock of Fc-T20 at a concentration of 2000 nM was prepared in buffer B. The stock was serially diluted to cover a range of 1 mM to 1000 nM by mixing the stock 1:1 with 6 nM IgG solution in the 96 well plate. The plates were rocked at room temperature and then incubated at 37° C. for two hours. The plates were aspirated and 100 μL/well of peroxidase conjugated F(ab¹)₂ goat anti-human Fc (Jackson Immuno Research West Grove, Pa.) diluted 1:10,000 in buffer B was added to each well. The plates were incubated 30 minutes at room temperature and then washed four times with 200 μL/well with ice cold buffer B. 100 μL tetramethylbenzidine peroxidase substrate (TMB) (Pierce, Rockford, Ill.) substrate solution was added to each well and incubated until color develops (5-10 minutes). 100 μL of TMB stop solution (KPL Gaithersburg, Md.) was added to each well and absorbance was measured in a spectrophotometer at 450 nM.

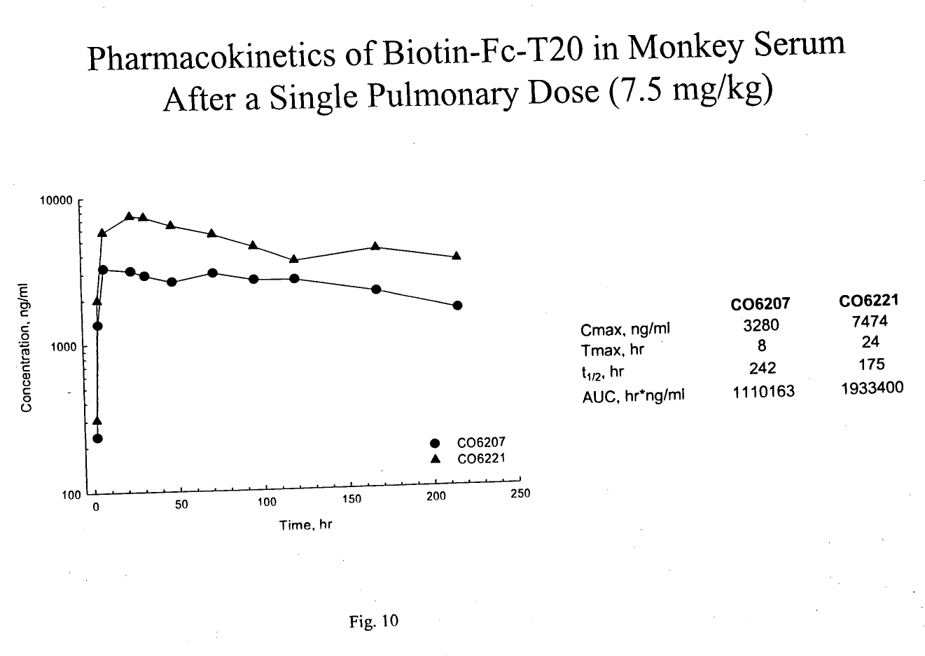

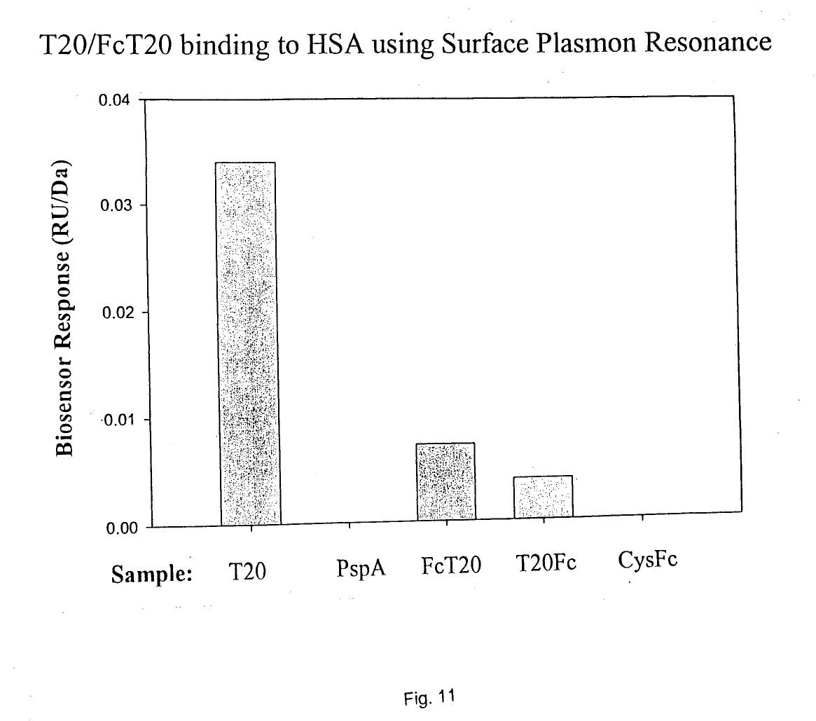

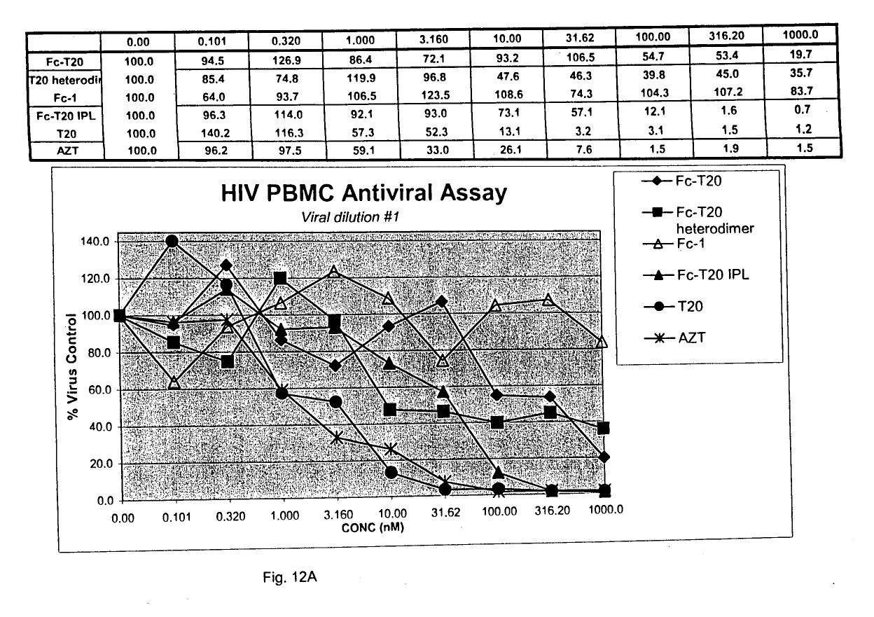

The results demonstrated sustained serum levels of biotin-FC-T20 with a t_{1/2} of 242 hours and 175 hours in each animal tested (FIG. 10).

### Example 15

### Chimeric protein Binding to Human Serum Albumin

Analysis of macromolecular interactions using surface plasmon resonance has been described in detail. Frostell-Karlsson et al. 2000, J. Med. Chem. 43:1986. A BIACORE 3000 instrument (Biacore AB Uppsala, Sweden) was used and all binding interactions were performed at 25° C. A carboxymethyl-modified dextran (CM5) sensor chip (Biacore. AB Uppsala, Sweden) was used for analysis. HAS (Albuminar) (Aventis Bridgewater, N.J.) was diluted to 100 μg/mL in 10 mM sodium acetate (pH 4.5) and immobilized to one flowcell of the sensor chip, using amine coupling as described. Frostell-Karlsson et al. 2000, J. Med. Chem. 43:1986. Final immobilization level was approximately 8500 Resonance Units (RU). A “mock-immobilized” surface using a separate flowcell was created using the same procedure in the absence of HSA and served as a reference for the binding studies.

Proteins or peptides (analyte) were diluted in HBS-N buffer (10 mM HEPES, pH 7.4; 150 mM NaCl) and injected over the HSA and reference surfaces for 3 minutes at a rate of 20 μL/min. After a 35 second dissociation phase, the surface was regenerated by a 30 second pulse of 10 mM glycine (pH 2.0) at a flow rate of 60 μL/min. Proteins and peptides were tested at concentrations of 100 μM, 10 μM, 1 μM, and 0.1 μM.

Human serum albumin binding of T20 was compared with human serum albumin binding of FcT20 and T20Fc. Negative controls include the bacterial protein Psp A and Cys-Fc which do not bind albumin.

The sensorgrams (Response Units versus time) generated for the mock-coated flowcell were automatically subtracted from the HSA-coated sensorgrams. Response at equilibrium (Req) was measured 30 seconds before the end of the injection phase and divided by the molecular weight of the analyte (Frostell-Karlsson et al. 2000, J. Med. Chem. 43:1986).

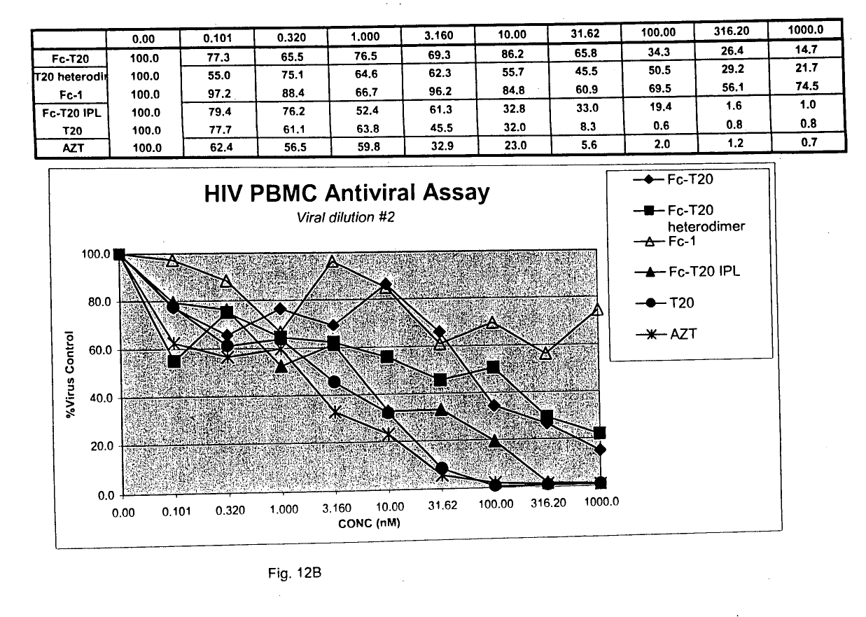

The results indicated binding of free T20 to human serum albumin was more than 3 fold greater than binding of either FcT20 or T20Fc to human serum albumin (FIG. 11).

### Example 16

### Antiviral Activity and Cellular Toxicity of Chemically Conjugated FC-T20 Measured in PBMCs

This assay was used to test the effectiveness of anti-HIV drug monotherapy in Peripheral Blood Mononuclear Cells (PBMC) infected with clinical or laboratory isolates of HIV-1. Drug efficacy was determined by the production of supernatant HIV-1 Reverse Transcriptase. Cytotoxicity is determined by the metabolic reduction of tetrazolium salts.

A. PBMC Isolation and Blasting

Peripheral blood monocular cells (PBMCs) were obtained from normal hepatitis and HIV-1 negative donors by ficoll-hypaque gradient separation. The mononuclear cells were washed to remove residual separation media, counted, viability determined and resuspended in RPMI 1640 medium supplemented with 15% FBS (heat inactivated), 2 mM L-glutamine, 100 U/mL penicillin, 100 μg/mL streptomycin, and 10 μg/mL gentamycin with 2 μg/mL phytohemagluttin (PHA) at 1×106 cells/mL. The cells were cultured for 48 to 72 hours at 37° C., 5% CO₂. Following incubation, cells were collected by centrifugation, washed and resuspended in RPMI 1640 supplemented with 15% FBS (heat inactivated), 2 mM L-glutamine, 100 U/mL penicillin, 100 μg/mL streptomycin, with 20 U/mL recombinant IL-2 (R & D Systems, Minneapolis, Minn.). IL-2 were included in the culture medium to maintain the cell division initiated by the PHA mitogenic stimulation. The cultures were then maintained until use by ½ culture volume change with fresh IL-2 containing medium every 3 days.

B. PBMC Assay

Human peripheral blood mononuclear cells from a minimum of three donors, that have been treated with PHA and IL-2, were counted, viability determined by Trypan Blue dye exclusion and mixed in equal ratios. Pooled donors were used to minimize the variability observed between individual donors which results from quantitative and qualitative differences in HIV infection and overall response to the PHA and IL-2 of primary lymphocyte populations. The cells were resuspended at 1×10⁶ cells/mL in RPMI 1640 without phenol red supplemented with 15% Fetal Bovine Serum (heat inactivated), 2 mM L-glutamine, 100 U/mL penicillin, 100 μg/mL streptomycin, 10 μg/mL gentamycin and IL-2 (20 U/mL, R&D Systems, Minneapolis, Minn.). Fifty microliters of cells were then distributed to the inner 60 wells of a 96 well round bottom microtiter culture plate in a standard format developed by the Infectious Disease Research department of Southern Research Institute (Frederick, Md.) Each plate contained cell control wells (cells only), virus control wells (cells plus virus), and experimental wells (drug plus cells plus virus). Serially diluted compounds were added to the microtiter plate followed by the appropriate pre-titered strains of HIV-1. All samples were assayed in triplicate with a replicate plate without virus for the determination of compound toxicity. The final volume per well is 200 μL. The assay was incubated for 6 days in a humidified atmosphere at 37° C., 5% CO₂, after which supernatants were collected, for analysis of RT activity and cell viability determined by MTS dye reduction. Wells were also examined microscopically and any abnormalities noted.

C. Test Plates

Compounds were maintained and diluted in aqueous solutions (medium) unless otherwise instructed. Nine half-log or log serial dilutions were made in tissue culture media (RPMI 1640 without phenol red, supplemented with 10% heat inactivated fetal bovine serum, 1% L-glutamine, 1% Penicillin/Streptomycin, and 50 μg/mL gentimycin). Virus and cell controls along with cell-free drug controls (control for drug color effects) were included on each test plate. In this assay drugs were used to pre-treat target cells for 30 minutes prior to the addition of virus. Virus was added and the infection allowed to proceed in the presence of drug for 45-60 minutes. Drugs and virus were aspirated, the cultures re-fed with drug or medium (controls) and the plates re-incubated at 37° C. in Air+5% CO₂. Cell, drug and virus containing cultures were incubated for seven (7) days with an interim feeding with drug and or medium at day 3 or 4.

D. Reverse Transcriptase Assay for Culture Supernatants

Reverse transcriptase activity was measured in cell-free supernatants. Tritiated thymidine triphosphate (NEN) (TTP) was resuspended in distilled H₂O at 5 Ci/mL. Poly rA and oligo dT were prepared as a stock solution which were kept at −20° C. The RT reaction buffer was prepared fresh on a daily basis and consisted of 125 μL 1.0 M EGTA, 125 μL dH₂O, 110 μL 10% SDS, 50 μL 1.0 M Tris (pH 7.4), 50 μL 1.0 M DTT, and 40 μL 1.0 M MgCl₂. These three solutions were mixed together in a ratio of 2 parts TTP, 1 part poly rA:oligo dT, and 1 part reaction buffer. Ten microliters of this reaction mixture was placed in a round bottom microtiter plate and 15 μL of virus containing supernatant was added and mixed. The plate was incubated at 37° C. in a water bath with a solid support to prevent submersion of the plate and incubated for 60 minutes. Following reaction, the reaction volume was spotted onto pieces of DE81 paper, washed 5 times for 5 minutes each in a 5% sodium phosphate buffer, 2 times for 1 minute each in distilled water, 2 times for 1 minute each in 70% ethanol, and then dried. Opti-Fluor 0 was added to each sample and incorporated radioactivity was quantitated utilizing a Wallac 1450 Microbetaplus liquid scintillation counter. RT activity was reported in counts per minute (cpm) for the analyzed sample volume.

E. MTS Staining for Cell Viability

At assay termination the assay plates were stained with the soluble tetrazolium-based dye MTS (CellTiter96® Reagent)(Promega Madison, Wis.) to determine cell viability and quantify compound toxicity. MTS is metabolized by the mitochondria enzymes of metabolically active cells to a soluble formazan product, allowing the rapid quantitative analysis cell viability and compound cytotoxicity. This reagent is a single stable solution that does not require preparation before use. At termination of the assay, 20 μL of MTS reagent was added per well and incubated for 4 hours at 37° C. Adhesive plate sealers were used in place of the lids, the sealed plate was inverted several times to mix the soluble formazan product and the plate was read spectrophotometrically at 490 nm with a Molecular Devices Vmax plate reader (Molecular Devices, Sunnyvale, Calif.).

F. Data Analysis

Using an in-house computer program, IC₅₀ (50% inhibition of virus replication, respectively) TC₅₀ (50% reduction in cell viability) and a therapeutic index (TI, TC₅₀/IC₅₀) were provided. The reverse transcriptase inhibitor AZT will be provided as a positive controls for all antiviral assays.

The results presented in FIGS. 12A and 12B demonstrate chimeric proteins comprised of T20 and Fc possess potent anti-viral activity against HIV.

All references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

1. A chimeric protein comprising at least a portion of an immunoglobulin constant region and at least one HIV fusion inhibitor.

2. The chimeric protein of claim 1, wherein the portion of an immunoglobulin constant region is a portion of an IgG constant region or fragment thereof.

3. The chimeric protein of claim 1, wherein the portion of an immunoglobulin constant region is an Fc fragment.

4. The chimeric protein of claim 1, wherein the portion of an immunoglobulin constant region is an FcRn binding partner.

5. The chimeric protein of claim 3, wherein the HIV fusion inhibitor is a peptide.

6. The chimeric protein of claim 5, wherein the HIV fusion inhibitor is a peptide comprising about 3-36 amino acids.

7. The chimeric protein of claim 1, wherein the HIV fusion inhibitor is a, a peptide, identified by a computer algorithm as an HIV fusion inhibitor.

8. The chimeric protein of claim 7, wherein the computer algorithm is chosen from ALLMOTI5, 107×178×4, or PLZIP.

9. The chimeric protein of claim 5, wherein the peptide is T20 (SEQ ID NO:1), or T21 (SEQ ID NO:2), or T1249 (SEQ ID NO:3).

10. The chimeric protein of claim 5, wherein the peptide is an analog of T20, T21, or T1249.

11. A pharmaceutical composition comprising the chimeric protein of any one of claim 1, 4, 5, or 9 and a pharmaceutically acceptable excipient.

12. The chimeric protein of claim 3, wherein the Fc fragment is an IgG1 Fc fragment.

13. The chimeric protein of claim 1 comprising the formula
F-L-I
wherein F is an Fc fragment of an immunoglobulin, L is a linker or a direct bond and I is an HIV fusion inhibitor.

14. The chimeric protein of claim 1 comprising the formula
I-L-F
wherein I is an HIV fusion inhibitor, L is a linker or a direct bond.

15. The chimeric protein of claim 13 or 14, wherein F comprises an amino acid sequence having at least 80% identity with the amino acid sequence at least 80% identity with the amino acid sequence set forth in SEQ ID NO:16.

16. The chimeric protein of claim 13 or 14, wherein F comprises an amino acid sequence having at least 80% identity with the amino acid sequence set forth in SEQ ID NO:17.

17. The chimeric protein of claim 12 or 13, wherein I is a peptide comprising about 3 to 36 amino acids.

18. The chimeric protein of claim 12 or 13, wherein L is a linker comprising about 1 to 20 amino acids.

19. The chimeric protein of claim 12 or 13, wherein L is a linker comprising about 1 to 10 amino acids.

20. The chimeric protein of claim 12 or 13, wherein L is a linker comprising about 1 to 5 amino acids.

21. The chimeric protein of claim 12 or 13, wherein L is a linker comprising the sequence -(Gly)ₙ-, wherein n is an integer of about 1 to about 10.

22. The chimeric protein of claim 12 or 13, wherein L is a linker comprising the sequence -(GGS)ₙ-, wherein n is an integer of about 1 to about 10.

23. The chimeric protein of claim 13 or 14, wherein I is T20 (SEQ ID NO:1), or T21 (SEQ ID NO:2), or T1249 (SEQ ID NO:3).

24. The chimeric protein of claim 13 or 14, wherein L is GGG, or SGGSGGS, or GGSGGSGGSGGSGG, or FC.

25. The chimeric protein of claim 1 comprising the formula
A-F-L-I
wherein A is a first linker or an affinity tag, F is an Fc fragment of an immunoglobulin, L is a second linker and I is an HIV fusion inhibitor.

26. The chimeric protein of claim 25, wherein A is MG(H)₁₀ SSGHIDDDDKHM and L is F-C.

27. The chimeric protein of claim 14, wherein the chimeric protein comprises

| | |
|---|---|
| EPKSSDKTHTCPPCPAPELLGGPSVFLFPPKPKDTLMISRTPEVTCVVVDVSHEDPEV | (SEQ ID NO: 4) |
| KFNWYVDGVEVHNAKTKPREEQYNSTYRVVSVLTVLHQDWLNGKEYKCKVSNKALP | |
| APIEKTISKAKGQPREPQVYTLPPSRDELTKNQVSLTCLVKGFYPSDIAVEWESNGQPE | |
| NNYKTTPPVLDSDGSFFLYSKLTVDKSRWQQGNFSCSVMHEALHNHYTQKSLSLSGG | |
| GYTSLIHSLIEESQNQQEKNEQELLELDKWASLWNWF. | |

28. The chimeric protein of claim 14, wherein the chimeric protein comprises

| | |
|---|---|
| YTSLIHSLIEESQNQQEKNEQELLELDKWASLWNWFSGGSGGSDTSHTCPPCPAPEL | (SEQ ID NO: 5) |
| LGGPSVFLFPPKPKDTLMISRTPEVTCVVVDVSHEDPEVKFNWYVDGVEVHNAKTKPR | |
| EEQYNSTYRVVSVLTVLHQDWLNGKEYKCKVSNKALPAPIEKTISKAKGQPREPQVYT | |
| LPPSRDELTKNQVSLTCLVKGFYPSDIAVEWESNGQPENNYKTTPPVLDSDGSFFLYS | |
| KLTVDKSRWQQGNVFSCSVMHEALHNHYTQKSLSLSPGK. | |

29. The chimeric protein of claim 14, wherein the chimeric protein comprises

| | |
|---|---|
| EPKSSDKTHTCPPCPAPELLGGPSVFLFPPKPKDTLMISRTPEVTCVVVDVSHEDPEV | (SEQ ID NO: 6) |
| KFNWYVDGVEVHNAKTKPREEQYNSTYRVVSVLTVLHQDWLNGKEYKCKVSNKALP | |
| APIEKTISKAKGQPREPQVYTLPPSRDELTKNQVSLTCLVKGFYPSDIAVEWESNGQPE | |
| NNYKTTPPVLDSDGSFFLYSKLTVDKSRWQQGNVFSCSVMHEALHNHYTQKSLSLSP | |
| GGGSGGSGGSGGSGGGYTSLIHSLIEESQNQQEKNEQELLELDKWASLWNWF. | |

30. The chimeric protein of claim 14, wherein the chimeric protein comprises

| | |
|---|---|
| YTSLIHSLIEESQNQQEKNEQELLELDKWASLWNWFGGSGGSGGSGGSGGSGGSDT | (SEQ ID NO: 7) |
| SHTCPPCPAPELLGGPSVFLFPPKPKDTLMISRTPEVTCVVVDVSHEDPEVKFNWYVD | |
| GVEVHNAKTKPREEQYNSTYRVVSVLTVLHQDWLNGKEYKCKVSNKALPAPIEKTISK | |
| AKGQPREPQVYTLPPSRDELTKNQVSLTCLVKGFYPSDIAVEWESNGQPENNYKTTP | |
| PVLDSDGSFFLYSKLTVDKSRWQQGNVFSCSVMHEALHNHYTQKSLSLSPGK. | |

31. The chimeric protein of claim 14, wherein the chimeric protein comprises

| | |
|---|---|
| EPKSSDKTHTCPPCPAPELLGGPSVFLFPPKPKDTLMISRTPEVTCVVVDVSHEDPEV | (SEQ ID NO: 8) |
| KFNWYVDGVEVHNAKTKPREEQYNSTYRVVSVLTVLHQDWLNGKEYKCKVSNKALP | |
| APIEKTISKAKGQPREPQVYTLPPSRDELTKNQVSLTCLVKGFYPSDIAVEWESNGQPE | |
| NNYKTTPPVLDSDGSFFLYSKLTVDKSRWQQGNVFSCSVMHEALHNHYTQKSLSLSP | |
| GFCYTSLIHSLIEESQNQQEKNEQELLELDKWASLWNWF. | |

32. The chimeric protein of claim 25, wherein the chimeric protein comprises

| | |
|---|---|
| MGHHHHHHHHHHSSGHIDDDDKHMEPKSSDKTHTCPPCPAPELLGGPSVFLFPPKPK | (SEQ ID NO: 9) |
| DTLMISRTPEVTCVVVDVSHEDPEVKFNWYVDGVEVHNAKTKPREEQYNSTYRVVSV | |
| LTVLHQDWLNGKEYKCKVSNKALPAPIEKTISKAKGQPREPQVYTLPPSRDELTKNQV | |
| SLTCLVKGFYPSDIAVEWESNGQPENNYKTTPPVLDSDGSFFLYSKLTVDKSRWQQG | |
| NVFSCSVMHEALHNHYTQKSLSLSPGFCYTSLIHSLIEESQNQQEKNEQELLELDKWA | |
| SLWNWF. | |

33. The chimeric protein of claim 1, wherein the chimeric protein has an in vivo half life of 24 hours.

34. The chimeric protein of claim 1, wherein the chimeric protein binds less serum albumin compared to the HIV fusion inhibitor prior to fusion with at least a portion of an immunoglobulin constant region.

35. The chimeric protein of claim 1, wherein the chimeric protein inhibits HIV infection of a cell.

36. A method of treating a subject infected with HIV comprising administering a therapeutically effective amount of at least one chimeric protein, wherein said chimeric protein comprises at least a portion of an immunoglobulin constant region linked to an HIV fusion inhibitor.

37. The method of claim 36, wherein the portion of an immunoglobulin constant region is an Fc fragment.

38. The method of claim 36, wherein the portion of an immunoglobulin constant region is an FcRn binding partner.

39. The method of claim 36 or 37, wherein the HIV fusion inhibitor is T20 (SEQ ID NO:1), or T21 (SEQ ID NO:2), or T1249 (SEQ ID NO:3).

40. The method of claim 36 or 37, wherein the chimeric protein comprises

| | |
|---|---|
| EPKSSDKTHTCPPCPAPELLGGPSVFLFPPKPKDTLMISRTPEVTCVVVDVSHEDPEV | (SEQ ID NO: 4) |
| KFNWYVDGVEVHNAKTKPREEQYNSTYRVVSVLTVLHQDWLNGKEYKCKVSNKALP | |
| APIEKTISKAKGQPREPQVYTLPPSRDELTKNQVSLTCLVKGFYPSDIAVEWESNGQPE | |
| NNYKTTPPVLDSDGSFFLYSKLTVDKSRWQQGNFSCSVMHEALHNHYTQKSLSLSGG | |
| GYTSLIHSLIEESQNQQEKNEQELLELDKWASLWNWF. | |

41. The method of claim 36 or 37, wherein the chimeric protein comprises

| | |
|---|---|
| YTSLIHSLIEESQNQQEKNEQELLELDKWASLWNWFSGGSGGSDTSHTCPPCPAPEL | (SEQ ID NO: 5) |
| LGGPSVFLFPPKPKDTLMISRTPEVTCVVVDVSHEDPEVKFNWYVDGVEVHNAKTKPR | |
| EEQYNSTYRVVSVLTVLHQDWLNGKEYKCKVSNKALPAPIEKTISKAKGQPREPQVYT | |
| LPPSRDELTKNQVSLTCLVKGFYPSDIAVEWESNGQPENNYKTTPPVLDSDGSFFLYS | |
| KLTVDKSRWQQGNVFSCSVMHEALHNHYTQKSLSLSPGK. | |

42. The method of claim 36 or 37, wherein the chimeric protein comprises

| | |
|---|---|
| EPKSSDKTHTCPPCPAPELLGGPSVFLFPPKPKDTLMISRTPEVTCVVVDVSHEDPEV | (SEQ ID NO: 6) |
| KFNWYVDGVEVHNAKTKPREEQYNSTYRVVSVLTVLHQDWLNGKEYKCKVSNKALP | |
| APIEKTISKAKGQPREPQVYTLPPSRDELTKNQVSLTCLVKGFYPSDIAVEWESNGQPE | |
| NNYKTTPPVLDSDGSFFLYSKLTVDKSRWQQGNVFSCSVMHEALHNHYTQKSLSLSP | |
| GGGSGGSGGSGGSGGGYTSLIHSLIEESQNQQEKNEQELLELDKWASLWNWF. | |

43. The method of claim 36 or 37, wherein the chimeric protein comprises

| | |
|---|---|
| YTSLIHSLIEESQNQQEKNEQELLELDKWASLWNWFGGSGGSGGSGGSGGSGGSDT | (SEQ ID NO: 7) |
| SHTCPPCPAPELLGGPSVFLFPPKPKDTLMISRTPEVTCVVVDVSHEDPEVKFNWYVD | |
| GVEVHNAKTKPREEQYNSTYRVVSVLTVLHQDWLNGKEYKCKVSNKALPAPIEKTISK | |
| AKGQPREPQVYTLPPSRDELTKNQVSLTCLVKGFYPSDIAVEWESNGQPENNYKTTP | |
| PVLDSDGSFFLYSKLTVDKSRWQQGNVFSCSVMHEALHNHYTQKSLSLSPGK. | |

44. The method of claim 36 or 37, wherein the chimeric protein comprises

| | |
|---|---|
| EPKSSDKTHTCPPCPAPELLGGPSVFLFPPKPKDTLMISRTPEVTCVVVDVSHEDPEV | (SEQ ID NO: 8) |
| KFNWYVDGVEVHNAKTKPREEQYNSTYRVVSVLTVLHQDWLNGKEYKCKVSNKALP | |
| APIEKTISKAKGQPREPQVYTLPPSRDELTKNQVSLTCLVKGFYPSDIAVEWESNGQPE | |
| NNYKTTPPVLDSDGSFFLYSKLTVDKSRWQQGNVFSCSVMHEALHNHYTQKSLSLSP | |
| GFCYTSLIHSLIEESQNQQEKNEQELLELDKWASLWNWF. | |

45. The method of claim 36 or 37, wherein the chimeric protein comprises

| | |
|---|---|
| MGHHHHHHHHHHSSGHIDDDDKHMEPKSSDKTHTCPPCPAPELLGGPSVFLFPPKPK | (SEQ ID NO: 9) |
| DTLMISRTPEVTCVVVDVSHEDPEVKFNWYVDGVEVHNAKTKPREEQYNSTYRVVSV | |
| LTVLHQDWLNGKEYKCKVSNKALPAPIEKTISKAKGQPREPQVYTLPPSRDELTKNQV | |
| SLTCLVKGFYPSDIAVEWESNGQPENNYKTTPPVLDSDGSFFLYSKLTVDKSRWQQG | |
| NVFSCSVMHEALHNHYTQKSLSLSPGFCYTSLIHSLIEESQNQQEKNEQELLELDKWA | |
| SLWNWF. | |

46. The method of claim 36 or 37, wherein the chimeric protein is administered intravenously, subcutaneously, nasally, orally, sublingually, rectally, vaginally, via aerosol, via pulmonary route or intramuscularly.

47. The method of claim 37, wherein the chimeric protein binds an FcRn.

48. The method of claim 36 or 37, wherein the chimeric protein is administered as a dosage of 1-5000 μg/kg one time per week.

49. The method of claim 36 or 37, wherein chimeric protein is administered 1-3 times a week.

50. A method of inhibiting HIV fusion with a mammalian cell comprising combining the mammalian cell with at least one chimeric protein, wherein said chimeric protein comprises at least a portion of an immunoglobulin constant region and an HIV fusion inhibitor.

51. The method of claim 50, wherein the portion of an immunoglobulin constant region is an Fc fragment.

52. The method of claim 50 or 51, wherein the HIV fusion inhibitor is T20 (SEQ ID NO:1), T21 (SEQ ID NO:2), or T1249 (SEQ ID NO:3).

53. The method of claim 50 or 51, wherein the chimeric protein comprises

| | |
|---|---|
| EPKSSDKTHTCPPCPAPELLGGPSVFLFPPKPKDTLMISRTPEVTCVVVDVSHEDPEV | (SEQ ID NO: 4) |
| KFNWYVDGVEVHNAKTKPREEQYNSTYRVVSVLTVLHQDWLNGKEYKCKVSNKALP | |
| APIEKTISKAKGQPREPQVYTLPPSRDELTKNQVSLTCLVKGFYPSDIAVEWESNGQPE | |
| NNYKTTPPVLDSDGSFFLYSKLTVDKSRWQQGNFSCSVMHEALHNHYTQKSLSLSGG | |
| GYTSLIHSLIEESQNQQEKNEQELLELDKWASLWNWF. | |

54. The method of claim 50 or 51, wherein the chimeric protein comprises

| | |
|---|---|
| YTSLIHSLIEESQNQQEKNEQELLELDKWASLWNWFSGGSGGSDTSHTCPPCPAPEL | (SEQ ID NO: 5) |
| LGGPSVFLFPPKPKDTLMISRTPEVTCVVVDVSHEDPEVKFNWYVDGVEVHNAKTKPR | |
| EEQYNSTYRVVSVLTVLHQDWLNGKEYKCKVSNKALPAPIEKTISKAKGQPREPQVYT | |
| LPPSRDELTKNQVSLTCLVKGFYPSDIAVEWESNGQPENNYKTTPPVLDSDGSFFLYS | |
| KLTVDKSRWQQGNVFSCSVMHEALHNHYTQKSLSLSPGK. | |

55. The method of claim 50 or 51, wherein the chimeric protein comprises

| | |
|---|---|
| EPKSSDKTHTCPPCPAPELLGGPSVFLFPPKPKDTLMISRTPEVTCVVVDVSHEDPEV | (SEQ ID NO: 6) |
| KFNWYVDGVEVHNAKTKPREEQYNSTYRVVSVLTVLHQDWLNGKEYKCKVSNKALP | |
| APIEKTISKAKGQPREPQVYTLPPSRDELTKNQVSLTCLVKGFYPSDIAVEWESNGQPE | |
| NNYKTTPPVLDSDGSFFLYSKLTVDKSRWQQGNVFSCSVMHEALHNHYTQKSLSLSP | |
| GGGSGGSGGSGGSGGGYTSLIHSLIEESQNQQEKNEQELLELDKWASLWNWF. | |

56. The method of claim 50 or 51, wherein the chimeric protein comprises

| | |
|---|---|
| YTSLIHSLIEESQNQQEKNEQELLELDKWASLWNWFGGSGGSGGSGGSGGSGGSDT | (SEQ ID NO: 7) |
| SHTCPPCPAPELLGGPSVFLFPPKPKDTLMISRTPEVTCVVVDVSHEDPEVKFNWYVD | |
| GVEVHNAKTKPREEQYNSTYRVVSVLTVLHQDWLNGKEYKCKVSNKALPAPIEKTISK | |
| AKGQPREPQVYTLPPSRDELTKNQVSLTCLVKGFYPSDIAVEWESNGQPENNYKTTP | |
| PVLDSDGSFFLYSKLTVDKSRWQQGNVFSCSVMHEALHNHYTQKSLSLSPGK. | |

57. The method of claim 50 or 51, wherein the chimeric protein comprises

| | |
|---|---|
| EPKSSDKTHTCPPCPAPELLGGPSVFLFPPKPKDTLMISRTPEVTCVVVDVSHEDPEV | (SEQ ID NO: 8) |
| KFNWYVDGVEVHNAKTKPREEQYNSTYRVVSVLTVLHQDWLNGKEYKCKVSNKALP | |
| APIEKTISKAKGQPREPQVYTLPPSRDELTKNQVSLTCLVKGFYPSDIAVEWESNGQPE | |
| NNYKTTPPVLDSDGSFFLYSKLTVDKSRWQQGNVFSCSVMHEALHNHYTQKSLSLSP | |
| GFCYTSLIHSLIEESQNQQEKNEQELLELDKWASLWNWF. | |

58. The method of claim 50 or 51, wherein the chimeric protein comprises

| | |
|---|---|
| MGHHHHHHHHHHSSGHIDDDDKHMEPKSSDKTHTCPPCPAPELLGGPSVFLFPPKPK | (SEQ ID NO: 9) |
| DTLMISRTPEVTCVVVDVSHEDPEVKFNWYVDGVEVHNAKTKPREEQYNSTYRVVSV | |
| LTVLHQDWLNGKEYKCKVSNKALPAPIEKTISKAKGQPREPQVYTLPPSRDELTKNQV | |
| SLTCLVKGFYPSDIAVEWESNGQPENNYKTTPPVLDSDGSFFLYSKLTVDKSRWQQG | |
| NVFSCSVMHEALHNHYTQKSLSLSPGFCYTSLIHSLIEESQNQQEKNEQELLELDKWA | |
| SLWNWF. | |

59. A method of making a chimeric protein comprising at least a portion of an immunoglobulin constant region linked to an HIV fusion inhibitor, comprising
a) transfecting a cell with a DNA construct comprising a first DNA sequence encoding at least a portion of immunoglobulin linked to a second DNA sequence encoding an HIV fusion inhibitor;
b) culturing said cell under conditions such that the chimeric protein is expressed; and
c) isolating said chimeric protein.

60. The method of claim 59, wherein the chimeric protein is made in a prokaryotic cell.

61. The method of claim 60, wherein the prokaryotic cell is E. coli.

62. The method of claim 59, wherein the chimeric protein is made in a eukaryotic cell.

63. The method of claim 59, wherein the DNA construct comprises a DNA sequence encoding an Fc fragment of an immunoglobulin.

64. The method of claim 59, wherein the DNA construct comprises a DNA sequence encoding a FcRn binding partner.

65. The method of claim 59, wherein the DNA construct comprises

| | |
|---|---|
| Gaaccaaagagctccgacaaaactcacacatgcccaccgtgcccagcacctgaactcctggggggaccgtcagtcttc | (SEQ ID NO: 10) |
| ctcttccccccaaaacccaaggacaccctcatgatctcccggacccctgaggtcacatgcgtggtggtggacgtgagcca | |
| cgaagaccctgaggtcaagttcaactggtacgtggacggcgtggaggtgcataatgccaagacaaagccgcgggagg | |
| agcagtacaacagcacgtaccgtgtggtcagcgtcctcaccgtcctgcaccaggactggctgaatggcaaggagtaca | |
| agtgcaaggtctccaacaaagccctcccagcccccatcgagaaaaccatctccaaagccaaagggcagccccgaga | |
| accacaggtgtacaccctgcccccatcccgggatgagctgaccaagaaccaggtcagcctgacctgcctggtcaaagg | |
| cttctatcccagcgacatcgccgtggagtgggagagcaatgggcagccggagaacaactacaagaccacgcctcccgt | |
| gttggactccgacggctccttcttcctctacagcaagctcaccgtggacaagagcaggtggcagcaggggaacgtcttctc | |
| atgctccgtgatgcatgaggctctgcacaaccactacacgcagaagagcctctcgctgagcggcggtggctacacttccc | |
| tgatccacagcctgatcgaagaatctcagaaccagcaggaaaagaacgaacaggaactgctcgagctggacaaatg | |
| ggcctctctgtggaactggttctga. | |

66. The method of claim 59, wherein the DNA construct comprises

| | |
|---|---|
| tacacttccctgatccacagcctgatcgaagaatctcagaaccagcaggaaaagaacgaacaggaactgctcgagctg | (SEQ ID NO: 11) |
| gacaaatgggcctctctgtggaactggttctccggaggcagcggcggctccgatactagtcacacatgcccaccgtgccc | |
| agcacctgaactcctggggggaccgtcagtcttcctcttccccccaaaacccaaggacaccctcatgatctcccggaccc | |
| ctgaggtcacatgcgtggtggtggacgtgagccacgaagaccctgaggtcaagttcaactggtacgtggacggcgtgga | |
| ggtgcataatgccaagacaaagccgcgggaggagcagtacaacagcacgtaccgtgtggtcagcgtcctcaccgtcct | |
| gcaccaggactggctgaatggcaaggagtacaagtgcaaggtctccaacaaagccctcccagcccccatcgagaaaa | |
| ccatctccaaagccaaagggcagccccgagaaccacaggtgtacaccctgcccccatcccgggatgagctgaccaag | |
| aaccaggtcagcctgacctgcctggtcaaaggcttctatcccagcgacatcgccgtggagtgggagagcaatgggcag | |
| ccggagaacaactacaagaccacgcctcccgtgttggactccgacggctccttcttcctctacagcaagctcaccgtgga | |
| caagagcaggtggcagcaggggaacgtcttctcatgctccgtgatgcatgaggctctgcacaaccactacacgcagaa | |
| gagcctctccctgtctccgggtaaatga. | |

67. The method of claim 59, wherein the DNA construct comprises

| | |
|---|---|
| gaaccaaagagctccgacaaaactcacacatgcccaccgtgcccagcacctgaactcctggggggaccgtcagtcttc | (SEQ ID NO: 12) |
| ctcttccccccaaaacccaaggacaccctcatgatctcccggacccctgaggtcacatgcgtggtggtggacgtgagcca | |
| cgaagaccctgaggtcaagttcaactggtacgtggacggcgtggaggtgcataatgccaagacaaagccgcgggagg | |
| agcagtacaacagcacgtaccgtgtggtcagcgtcctcaccgtcctgcaccaggactggctgaatggcaaggagtaca | |
| agtgcaaggtctccaacaaagccctcccagcccccatcgagaaaaccatctccaaagccaaagggcagccccgaga | |
| accacaggtgtacaccctgcccccatcccgggatgagctgaccaagaaccaggtcagcctgacctgcctggtcaaagg | |
| cttctatcccagcgacatcgccgtggagtgggagagcaatgggcagccggagaacaactacaagaccacgcctcccgt | |
| gttggactccgacggctccttcttcctctacagcaagctcaccgtggacaagagcaggtggcagcaggggaacgtcttctc | |
| atgctccgtgatgcatgaggctctgcacaaccactacacgcagaagagcctctcgctgagccccggtggcggttccggc | |
| ggctccggcggctctggtggctctggcggtggctacacttccctgatccacagcctgatcgaagaatctcagaaccagca | |
| ggaaaagaacgaacaggaactgctcgagctggacaaatgggcctctctgtggaactggttctga. | |

68. The method of claim 59, wherein the DNA construct comprises

| | |
|---|---|
| tacacttccctgatccacagcctgatcgaagaatctcagaaccagcaggaaaagaacgaacaggaactgctc | (SEQ ID NO: 13) |
| gagctggacaaatgggcctctctgtggaactggttcggtggcagcggtggtagcggcggtagcggcggttccggaggca | |
| gcggcggctccgatactagtcacacatgcccaccgtgcccagcacctgaactcctggggggaccgtcagtcttcctcttcc | |
| ccccaaaacccaaggacaccctcatgatctcccggacccctgaggtcacatgcgtggtggtggacgtgagccacgaag | |
| accctgaggtcaagttcaactggtacgtggacggcgtggaggtgcataatgccaagacaaagccgcgggaggagcag | |
| tacaacagcacgtaccgtgtggtcagcgtcctcaccgtcctgcaccaggactggctgaatggcaaggagtacaagtgca | |
| aggtctccaacaaagccctcccagcccccatcgagaaaaccatctccaaagccaaagggcagccccgagaaccaca | |
| ggtgtacaccctgcccccatcccgggatgagctgaccaagaaccaggtcagcctgacctgcctggtcaaaggcttctatc | |
| ccagcgacatcgccgtggagtgggagagcaatgggcagccggagaacaactacaagaccacgcctcccgtgttgga | |
| ctccgacggctccttcttcctctacagcaagctcaccgtggacaagagcaggtggcagcaggggaacgtcttctcatgctc | |
| cgtgatgcatgaggctctgcacaaccactacacgcagaagagcctctccctgtctccgggtaaatga. | |

69. The method of claim 59, wherein the DNA construct comprises

| | |
|---|---|
| gaaccaaagagctccgacaaaactcacacatgcccaccgtgcccagcacctgaactcctggggggaccgtc | (SEQ ID NO: 14) |
| agtcttcctcttccccccaaaacccaaggacaccctcatgatctcccggacccctgaggtcacatgcgtggtggtggacgt | |
| gagccacgaagaccctgaggtcaagttcaactggtacgtggacggcgtggaggtgcataatgccaagacaaagccgc | |
| gggaggagcagtacaacagcacgtaccgtgtggtcagcgtcctcaccgtcctgcaccaggactggctgaatggcaagg | |
| agtacaagtgcaaggtctccaacaaagccctcccagcccccatcgagaaaaccatctccaaagccaaagggcagccc | |
| cgagaaccacaggtgtacaccctgcccccatcccgggatgagctgaccaagaaccaggtcagcctgacctgcctggtc | |
| aaaggcttctatcccagcgacatcgccgtggagtgggagagcaatgggcagccggagaacaactacaagaccacgc | |
| ctcccgtgttggactccgacggctccttcttcctctacagcaagctcaccgtggacaagagcaggtggcagcaggggaac | |
| gtcttctcatgctccgtgatgcatgaggctctgcacaaccactacacgcagaagagcctctcgctgagcccgggcttttgct | |
| acacttccctgatccacagcctgatcgaagaatctcagaaccagcaggaaaagaacgaacaggaactgctcgagctg | |
| gacaaatgggcctctctgtggaactggttctga. | |

70. The method of claim 58, wherein the DNA construct comprises

| | |
|---|---|
| atgggccatcatcatcatcatcatcatcatcatcacagcagcggccatatcgacgacgacgacaagcatatggaaccaa | (SEQ ID NO: 15) |
| agagctccgacaaaactcacacatgcccaccgtgcccagcacctgaactcctggggggaccgtcagtcttcctcttcccc | |
| ccaaaacccaaggacaccctcatgatctcccggacccctgaggtcacatgcgtggtggtggacgtgagccacgaagac | |
| cctgaggtcaagttcaactggtacgtggacggcgtggaggtgcataatgccaagacaaagccgcgggaggagcagta | |
| caacagcacgtaccgtgtggtcagcgtcctcaccgtcctgcaccaggactggctgaatggcaaggagtacaagtgcaa | |
| ggtctccaacaaagccctcccagcccccatcgagaaaaccatctccaaagccaaagggcagccccgagaaccacag | |
| gtgtacaccctgcccccatcccgggatgagctgaccaagaaccaggtcagcctgacctgcctggtcaaaggcttctatcc | |
| cagcgacatcgccgtggagtgggagagcaatgggcagccggagaacaactacaagaccacgcctcccgtgttggact | |
| ccgacggctccttcttcctctacagcaagctcaccgtggacaagagcaggtggcagcaggggaacgtcttctcatgctcc | |
| gtgatgcatgaggctctgcacaaccactacacgcagaagagcctctcgctgagcccgggcttttgctacacttccctgatc | |
| cacagcctgatcgaagaatctcagaaccagcaggaaaagaacgaacaggaactgctcgagctggacaaatgggcct | |
| ctctgtggaactggttc. | |

71. The method of claim 62, wherein the eukaryotic cell is a CHO cell.

72. A kit for the diagnosis of an HIV infection comprising a chimeric protein and a container, wherein said chimeric protein comprises at least a portion of immunoglobulin linked to an HIV fusion inhibitor.

73. A kit for testing drug sensitivity of an HIV specimen comprising a chimeric protein and a container, wherein said chimeric protein comprises at least a portion of an immunoglobulin constant region linked to an HIV fusion inhibitor.

74. The chimeric protein of claim 1, wherein said chimeric protein is a dimer.

75. The chimeric protein of claim 1, wherein the dimer is a monomer/dimer hybrid comprising a first chain and a second chain, wherein said first chain comprises an Fc fragment of an immunoglobulin linked to an HIV fusion inhibitor and said second chain comprises an Fc fragment without an HIV fusion inhibitor linked to it.

76. A method of making a chimeric protein comprising an Fc fragment of an immunoglobulin linked to an HIV inhibitor, said method comprising
a) transfecting a cell with a DNA construct comprising a DNA sequence encoding an Fc fragment of an immunoglobulin linked to a DNA sequence encoding intein;
b) culturing said cell under conditions such that the Fc fragment and intein is expressed;
c) isolating said Fc fragment and intein from said cell;
d) chemically synthesizing an HIV fusion inhibitor having a cysteine on the N terminus;
e) reacting the isolated Fc of c) with MESNA to generate a C terminal thioester;
f) reacting the inhibitor of d) with the Fc of e) to make an Fc-fusion inhibitor chimeric protein.

77. A nucleic acid molecule comprising a first nucleic acid sequence encoding at least a portion of an immunoglobulin constant region operatively linked to a second DNA sequence encoding an HIV fusion inhibitor.

78. The nucleic acid molecule of claim 77, wherein the nucleic acid molecule is comprised of SEQ ID NO:10.

79. The nucleic acid molecule of claim 0.77, wherein the nucleic acid molecule is comprised of SEQ ID NO:11.

80. The nucleic acid molecule of claim 77, wherein the nucleic acid molecule is comprised of SEQ ID NO:12.

81. The nucleic acid molecule of claim 77, wherein the nucleic acid molecule is comprised of SEQ ID NO:13.

82. The nucleic acid molecule of claim 77, wherein the nucleic acid molecule is comprised of SEQ ID NO:14.

83. A vector comprising the nucleic acid molecule of claim 77, 78, 79, 80, 81, or 82.

84. The vector of claim 83, wherein said portion of an immunoglobulin constant region is an Fc fragment.

85. The vector of claim 83, wherein said portion of an immunoglobulin constant region is an FcRn binding partner.

86. The vector of claim 83, wherein an HIV fusion inhibitor is T20, T21 or T1249.

87. A host cell comprising the vector of claim 83.

88. The host cell of claim 87, wherein said host cell is a CHO cell.

89. A chimeric protein comprising an Fc fragment linked to an HIV fusion inhibitor made according to the method of claim 76.

90. A method of making a chimeric protein comprising at least a portion of an immunoglobulin constant region linked to an HIV fusion inhibitor, comprising
a) obtaining a transgenic animal expressing the chimeric protein; and
b) isolating said chimeric protein from said transgenic animal.

91. A chimeric protein comprising at least one FcRn binding partner and at least one HIV fusion inhibitor.

92. The chimeric protein of claim 91, wherein the FcRn binding partner is a peptide mimetic of an Fc fragment of an immunoglobulin.


SEQUENCE LISTING (59 sequences)

SEQ ID NO: 1 - protein, 36 aa, Human immunodeficiency virus
YTSLIHSLIEESQNQQEKNEQELLELDKWASLWNWF

SEQ ID NO: 2 - protein, 37 aa, Human immunodeficiency virus
NNLRAIEAQQHLLQLTVWGIKQLQARILAVERYLKDQ

SEQ ID NO: 3 - protein, 39 aa, Human immunodeficiency virus
WQEWEQKITALLEQAQIQQEKNEYELQKLDKWASLWEWF

SEQ ID NO: 4 - protein, 267 aa, Artificial Sequence
  Description of Artificial Sequence Synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNFSCSVMHEALHNHYTQKSLSLSGGGYTSLIHSLIEESQNQQEKNEQELLELDKWASLWNWF

SEQ ID NO: 5 - protein, 270 aa, Artificial Sequence
  Description of Artificial Sequence Synthetic construct
YTSLIHSLIEESQNQQEKNEQELLELDKWASLWNWFSGGSGGSDTSHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 282 aa, Artificial Sequence
  Description of Artificial Sequence Synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGSGGSGGSGGGYTSLIHSLIEESQNQQEKNEQELLELDKWASLWNWF

SEQ ID NO: 7 - protein, 281 aa, Artificial Sequence
  Description of Artificial Sequence Synthetic construct
YTSLIHSLIEESQNQQEKNEQELLELDKWASLWNWFGGSGGSGGSGGSGGSGGSDTSHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 269 aa, Artificial Sequence
  Description of Artificial Sequence Synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGFCYTSLIHSLIEESQNQQEKNEQELLELDKWASLWNWF

SEQ ID NO: 9 - protein, 293 aa, Artificial Sequence
  Description of Artificial Sequence Synthetic construct
MGHHHHHHHHHHSSGHIDDDDKHMEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGFCYTSLIHSLIEESQNQQEKNEQELLELDKWASLWNWF

SEQ ID NO: 10 - DNA, 807 bp, Artificial Sequence
  Description of Artificial Sequence Synthetic construct
gaaccaaagagctccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctcgctgagcggcggtggctacacttccctgatccacagcctgatcgaagaatctcagaaccagcaggaaaagaacgaacaggaactgctcgagctggacaaatgggcctctctgtggaactggttctga

SEQ ID NO: 11 - DNA, 813 bp, Artificial Sequence
  Description of Artificial Sequence Synthetic construct
tacacttccctgatccacagcctgatcgaagaatctcagaaccagcaggaaaagaacgaacaggaactgctcgagctggacaaatgggcctctctgtggaactggttctccggaggcagcggcggctccgatactagtcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - DNA, 849 bp, Artificial Sequence
  Description of Artificial Sequence Synthetic construct
gaaccaaagagctccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctcgctgagccccggtggcggttccggcggctccggcggctctggtggctctggcggtggctacacttccctgatccacagcctgatcgaagaatctcagaaccagcaggaaaagaacgaacaggaactgctcgagctggacaaatgggcctctctgtggaactggttctga

SEQ ID NO: 13 - DNA, 846 bp, Artificial Sequence
  Description of Artificial Sequence Synthetic construct
tacacttccctgatccacagcctgatcgaagaatctcagaaccagcaggaaaagaacgaacaggaactgctcgagctggacaaatgggcctctctgtggaactggttcggtggcagcggtggtagcggcggtagcggcggttccggaggcagcggcggctccgatactagtcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - DNA, 810 bp, Artificial Sequence
  Description of Artificial Sequence Synthetic construct
gaaccaaagagctccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctcgctgagcccgggcttttgctacacttccctgatccacagcctgatcgaagaatctcagaaccagcaggaaaagaacgaacaggaactgctcgagctggacaaatgggcctctctgtggaactggttctga

SEQ ID NO: 15 - DNA, 879 bp, Artificial Sequence
  Description of Artificial Sequence Synthetic construct
atgggccatcatcatcatcatcatcatcatcatcacagcagcggccatatcgacgacgacgacaagcatatggaaccaaagagctccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctcgctgagcccgggcttttgctacacttccctgatccacagcctgatcgaagaatctcagaaccagcaggaaaagaacgaacaggaactgctcgagctggacaaatgggcctctctgtggaactggttc

SEQ ID NO: 16 - protein, 228 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNFSCSVMHEALHNHYTQKSLSLS

SEQ ID NO: 17 - protein, 227 aa, Homo sapiens
DTSHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 102 aa, Human immunodeficiency virus
SGIVQQQNNLLRAIEAQQHLLQLTVWGIKQCCGRISGIVQQQNNLLRAIEQQHLLQLTVWGIKQLQARSGGRGGWMEWDREINNYTSLIHSLIEESQNQQEK

SEQ ID NO: 19 - protein, 213 aa, Human immunodeficiency virus
QLLSGIVQQQNNLRAIEAQQHLLQLTVWGIKQLQARILAGGSGGHTTWMEWDREINNYTSLIHSLIEESQNQQEKNEQELLEGSSGGQLLSGIVQQQNNLRAIEAQQHLLQLTVWGIKQLQARILAGGSGGHTTWMEWDREINNYTSLIHSLIEESQNQQEKNEQELLEGSSGGQLLSGIVQQQNNLRAIEAQQHLLQLTVWGIKQLQARILA

SEQ ID NO: 20 - protein, 238 aa, Artificial Sequence
  Description of Artificial Sequence Amino acid sequence of Fc-MESNA
MGIEGRGAAAVDTSHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGF

SEQ ID NO: 21 - DNA, 714 bp, Artificial Sequence
  Description of Artificial Sequence Nucleotide sequence of Fc CDS in pTWIN1
atgggcattgaaggcagaggcgccgctgcggtcgatactagtcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagtctctccctgtctccgggtttt

SEQ ID NO: 22 - protein, 11 aa, Artificial Sequence
  Description of Artificial Sequence Synthetic peptide
PKNSSMISNTP

SEQ ID NO: 23 - protein, 7 aa, Artificial Sequence
  Description of Artificial Sequence Synthetic peptide
HQSLGTQ

SEQ ID NO: 24 - protein, 8 aa, Artificial Sequence
  Description of Artificial Sequence Synthetic peptide
HQNLSDGK

SEQ ID NO: 25 - protein, 8 aa, Artificial Sequence
  Description of Artificial Sequence Synthetic peptide
HQNISDGK

SEQ ID NO: 26 - protein, 8 aa, Artificial Sequence
  Description of Artificial Sequence Synthetic peptide
VISSHLGQ

SEQ ID NO: 27 - protein, 3 aa, Artificial Sequence
  Description of Artificial Sequence Linker peptide
GGG

SEQ ID NO: 28 - protein, 7 aa, Artificial Sequence
  Description of Artificial Sequence Linker peptide
SGGSGGS

SEQ ID NO: 29 - protein, 18 aa, Artificial Sequence
  Description of Artificial Sequence Linker peptide
GGSGGSGGSGGSGGSGGS

SEQ ID NO: 30 - DNA, 61 bp, Artificial Sequence
  Description of Artificial Sequence Primer
gatcaggctgtggatcagggaagtgtagccaccgccacccggagacagggagaggcttttc

SEQ ID NO: 31 - DNA, 35 bp, Artificial Sequence
  Description of Artificial Sequence Primer
tcgcctgctcttccaacgccgacaaaactcacaca

SEQ ID NO: 32 - DNA, 62 bp, Artificial Sequence
  Description of Artificial Sequence Primer
gcagttcctgttcgttcttttcctgctggttctgagattcttcgatcaggctgtggatcagg

SEQ ID NO: 33 - DNA, 71 bp, Artificial Sequence
  Description of Artificial Sequence Primer
accaccctgcagtcagaaccagttccacagagaggcccatttgtccagctcgagcagttcctgttcgttct

SEQ ID NO: 34 - DNA, 29 bp, Artificial Sequence
  Description of Artificial Sequence Primer
ccggagacagggagaggcttttctgcgtg

SEQ ID NO: 35 - DNA, 60 bp, Artificial Sequence
  Description of Artificial Sequence Primer
ctggacaaatgggcctctctgtggaactggttcggcggtggtggcgacaaaactcacaca

SEQ ID NO: 36 - DNA, 63 bp, Artificial Sequence
  Description of Artificial Sequence Primer
gaagaatctcagaaccagcaggaaaagaacgaacaggaactgctcgagctggacaaatgggcc

SEQ ID NO: 37 - DNA, 28 bp, Artificial Sequence
  Description of Artificial Sequence Primer
ctactactgcagttaacccggagacagg

SEQ ID NO: 38 - DNA, 64 bp, Artificial Sequence
  Description of Artificial Sequence Primer
ggtggttgctcttccaactacacttccctgatccacagcctgatcgaagaatctcagaaccagc

SEQ ID NO: 39 - DNA, 34 bp, Artificial Sequence
  Description of Artificial Sequence Primer
ttttgaattctcagaaccagttccacagagaggc

SEQ ID NO: 40 - DNA, 32 bp, Artificial Sequence
  Description of Artificial Sequence Primer
tgtcgctgagcggcggtggctacacttccctg

SEQ ID NO: 41 - DNA, 36 bp, Artificial Sequence
  Description of Artificial Sequence Primer
gactcagacccgcgcctacacttccctgatccacag

SEQ ID NO: 42 - DNA, 28 bp, Artificial Sequence
  Description of Artificial Sequence Primer
tttttccggagaaccagttccacagaga

SEQ ID NO: 43 - DNA, 36 bp, Artificial Sequence
  Description of Artificial Sequence Primer
tattggcggccgccctggctccgactcagacccgcg

SEQ ID NO: 44 - DNA, 25 bp, Artificial Sequence
  Description of Artificial Sequence Primer
cgcacaccggccttcgcaattccaa

SEQ ID NO: 45 - DNA, 36 bp, Artificial Sequence
  Description of Artificial Sequence Primer
cggctccggcggctctggtggctctggcggtggcta

SEQ ID NO: 46 - DNA, 36 bp, Artificial Sequence
  Description of Artificial Sequence Primer
ctctcgctgagccccggtggcggttccggcggctcc

SEQ ID NO: 47 - DNA, 25 bp, Artificial Sequence
  Description of Artificial Sequence Primer
ctgctgttggcggccgccctggctc

SEQ ID NO: 48 - DNA, 36 bp, Artificial Sequence
  Description of Artificial Sequence Primer
ccgctaccaccgctgccaccgaaccagttccacaga

SEQ ID NO: 49 - DNA, 36 bp, Artificial Sequence
  Description of Artificial Sequence Primer
tcacatccggaaccgccgctaccgccgctaccaccg

SEQ ID NO: 50 - DNA, 37 bp, Artificial Sequence
  Description of Artificial Sequence Primer
agagctcgctgagcccgggcttttgctacacttccct

SEQ ID NO: 51 - DNA, 42 bp, Artificial Sequence
  Description of Artificial Sequence Primer
gtggtcatatgggcattgaaggcagaggcgccgctgcggtcg

SEQ ID NO: 52 - DNA, 50 bp, Artificial Sequence
  Description of Artificial Sequence Primer
ggtggttgctcttccgcaaaaacccggagacagggagagactcttctgcg

SEQ ID NO: 53 - DNA, 37 bp, Artificial Sequence
  Description of Artificial Sequence Primer
gctactgcagccaccatggccttgacctttgctttac

SEQ ID NO: 54 - DNA, 44 bp, Artificial Sequence
  Description of Artificial Sequence Primer
cagttccggagctgggcacggcggagagcccacagagcagcttg

SEQ ID NO: 55 - protein, 4 aa, Homo sapiens
ELLG

SEQ ID NO: 56 - protein, 10 aa, Artificial Sequence
  Description of Artificial Sequence Linker peptide
GGGGGGGGGG

SEQ ID NO: 57 - protein, 30 aa, Artificial Sequence
  Description of Artificial Sequence Linker peptide
GGSGGSGGSGGSGGSGGSGGSGGSGGSGGS

SEQ ID NO: 58 - protein, 15 aa, Artificial Sequence
  Description of Artificial Sequence Linker peptide
GGSGGSGGSGGSGGG

SEQ ID NO: 59 - protein, 3 aa, Artificial Sequence
  Description of Artificial Sequence Linker peptide
VGC